(19) United States
(12) Patent Application Publication
(10) Pub. No.: US 2019/0038178 A1
(43) Pub. Date: Feb. 7, 2019

(54) MAGNETIC FIELD SENSOR SYSTEM AND FLEXIBLE DEVICE INCLUDING THE SAME

(71) Applicant: OLYMPUS CORPORATION, Tokyo (JP)

(72) Inventors: Yasuo SASAKI, Machida-shi (JP); Hiromasa FUJITA, Hachioji-shi (JP); Eiji YAMAMOTO, Musashimurayama-shi (JP)

(73) Assignee: OLYMPUS CORPORATION, Tokyo (JP)

(21) Appl. No.: 16/152,490

(22) Filed: Oct. 5, 2018

Related U.S. Application Data
(63) Continuation of application No. PCT/JP2016/061158, filed on Apr. 5, 2016.

(51) Int. Cl. A61B 5/06; G01B 7/30; A61B 34/20

(52) U.S. Cl. CPC A61B 5/062; G01B 7/30; A61M 25/0127; A61B 2034/2051; A61B 2562/0223; A61B 34/20

(57) ABSTRACT

A magnetic field sensor system includes a one-axis magnetic field generator that generates a magnetic field, three or more three-axis magnetic field detectors that detect a magnetic field for each axis and are arranged on a substantially straight line, and a calculator that calculates, from a detection result of the magnetic field, a spatial position or a spatial direction of the one-axis magnetic field generator. The calculator selects two or more three-axis magnetic field detectors, which generate no symmetry of magnetic fields, based on a preset judgment standard of symmetry of magnetic fields, and calculates the spatial position or the spatial direction of the one-axis magnetic field generator, based on detection results of the magnetic field between the selected two or more three-axis magnetic field detectors and the one-axis magnetic field generator.

### CROSS-REFERENCE TO RELATED APPLICATION

This application is a Continuation Application of PCT Application No. PCT/JP2016/061158, filed Apr. 5, 2016, the entire contents of which are incorporated herein by reference.

### BACKGROUND OF THE INVENTION

### 1. Field of the Invention

The present invention relates to a magnetic field sensor system for determining at least one of the position and the direction of a magnetic field generator for generating a magnetic field or a magnetic field detector for detecting a magnetic field, and to a flexible device including such a magnetic field sensor system.

### 2. Description of the Related Art

International Publication No. 94/04938 discloses a method of determining the three-dimensional position of one magnetic field sensor. The magnetic field sensor is a magnetic field detector for detecting a magnetic field. That is, a system disclosed in the Publication includes the magnetic field sensor and a plurality of magnetic field generators. Each magnetic field generator has a plurality of magnetic field generation elements. Each magnetic field generator generates magnetic fields by the magnetic field generation elements. The magnetic field sensor measures these magnetic fields to determine the position of the magnetic field sensor from these measured data. The Publication discloses two techniques as a method of determining the position of a one-axis magnetic field sensor. A first technique uses a one-axis magnetic field sensor having a one-axis magnetic field detection element and three three-axis magnetic field generators each having three one-axis magnetic field generation elements, the one-axis being orthogonal to other two-axis. The one-axis magnetic field sensor measures magnetic fields generated by the three three-axis magnetic field generators to determine the position of the one-axis magnetic field sensor.

A second technique uses one three-axis magnetic field sensor having three one-axis magnetic field detection elements and one three-axis magnetic field generator having three one-axis magnetic field generation elements. The one three-axis magnetic field sensor measures magnetic field generated by the one three-axis magnetic field generator to determine the position of the one three-axis magnetic field sensor.

### BRIEF SUMMARY OF THE INVENTION

According to a first aspect of the invention, there is provided a magnetic field sensor system comprising: one of a one-axis magnetic field generator and a two-axis magnetic field generator that generates a magnetic field; three or more three-axis magnetic field detectors that detect a magnetic field for each axis and are arranged on a substantially straight line; and a calculator that calculates, from a detection result of the magnetic field, at least one of a spatial position and a spatial direction of the one of the one-axis magnetic field generator and the two-axis magnetic field generator, wherein the calculator is configured to select two or more three-axis magnetic field detectors, which generate no symmetry of magnetic fields, from among the three or more three-axis magnetic field detectors, based on a preset judgment standard of symmetry of magnetic fields, and calculate the at least one of the spatial position and the spatial direction of the one of the one-axis magnetic field generator and the two-axis magnetic field generator, based on detection results of the magnetic field between the selected two or more three-axis magnetic field detectors and the one of the one-axis magnetic field generator and the two-axis magnetic generator.

According to a second aspect of the invention, there is provided a flexible device comprising: the magnetic field sensor system according to the first aspect, and a flexible member, wherein the flexible member includes one of a plurality of one-axis magnetic field generators and a plurality of two-axis magnetic field generators, the flexible device further comprises a controller configured to generate a magnetic field from each of the plurality of the magnetic field generators at different times from each other and to cause the three or more three-axis magnetic field detectors to detect a magnetic field in time series, and the calculator is configured to calculate at least one of a spatial position and a spatial direction of each of the plurality of magnetic field generators, based on time-series detection results obtained by the three or more three-axis magnetic field detectors.

According to a third aspect of the invention, there is provided a magnetic field sensor system comprising: one of a one-axis magnetic field detector and a two-axis magnetic field detector that detects a magnetic field; three or more three-axis magnetic field generators that generate a magnetic field for each axis and are arranged on a substantially straight line; and a calculator that calculates, from a detection result of the magnetic field, at least one of a spatial position and a spatial direction of the one of the one-axis magnetic field detector and the two-axis magnetic field detector, wherein the calculator is configured to select two or more three-axis magnetic field generators, which generate no symmetry of magnetic fields, from among the three or more three-axis magnetic field generators, based on a preset judgment standard of symmetry of magnetic fields, and calculate the at least one of the spatial position and the spatial direction of the one of the one-axis magnetic field detector and the two-axis magnetic field detector, based on detection results of the magnetic field between the selected two or more three-axis magnetic field generators and the one of the one-axis magnetic field detector and the two-axis magnetic detector.

According to a fourth aspect of the invention, there is provided a flexible device comprising: the magnetic field sensor system according to the third aspect, and a flexible member, wherein the flexible member includes one of a plurality of one-axis magnetic field detectors and a plurality of two-axis magnetic field detectors, the flexible device further comprises a controller configured to generate a magnetic field from each of the three or more tree-axis magnetic field generators at different times from each other and to cause the plurality of magnetic field detectors to detect an axial magnetic field component in time series, and the calculator is configured to calculate at least one of a spatial position and a spatial direction of each of the plurality of magnetic field detectors, based on a detection result of time-series axial-direction magnetic field components obtained by the plurality of magnetic field detectors.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a schematic diagram for illustrating a configuration example of a magnetic field sensor system according to a first embodiment of the present invention.

FIG. 1B is a schematic diagram for illustrating another configuration example of the magnetic field sensor system according to the first embodiment.

FIG. 2 is a diagram for illustrating a magnetic field detected in the magnetic field sensor system according to the first embodiment.

FIG. 3 is a diagram for illustrating an arrangement of respective one-axis coils of a three-axis coil.

FIG. 4 is a diagram for illustrating a magnetic field detection/generation region.

FIG. 5 is a diagram for illustrating a configuration of an antenna in still another configuration example of the magnetic field sensor system according to the first embodiment.

FIG. 6 is a diagram for illustrating another configuration example of the magnetic field sensor system according to the first embodiment.

FIG. 7A is a diagram for illustrating a magnetic field generated by a one-axis coil.

FIG. 7B is a diagram in which FIG. 7A is fitted to a coordinate system.

FIG. 8 is a diagram for illustrating the case where magnetic fields have symmetry.

FIG. 9 is a flowchart for illustrating one example of the operation of the magnetic field sensor system according to the first embodiment in the case where magnetic fields have symmetry.

FIG. 10 is a flowchart for illustrating another example of the operation of the magnetic field sensor system according to the first embodiment in the case where magnetic fields have symmetry.

FIG. 11 is a schematic diagram for illustrating a configuration example of a magnetic field sensor system according to a second embodiment of the present invention.

FIG. 12 is a flowchart for illustrating one example of the operation of the magnetic field sensor system according to the second embodiment in the case where magnetic fields have symmetry.

FIG. 13 is a flowchart for illustrating another example of the operation of the magnetic field sensor system according to the second embodiment.

FIG. 14A is a diagram for illustrating the arrangement of a third three-axis coil.

FIG. 14B is a diagram for illustrating the arrangement of respective one-axis coils constituting each of three-axis coils in FIG. 14A.

FIG. 15 is a schematic diagram for illustrating a configuration example of a magnetic field sensor system according to a third embodiment of the present invention.

FIG. 16 is a flowchart for illustrating one example of the operation of the magnetic field sensor system according to the third embodiment.

FIG. 17 is a flowchart for illustrating one example of the operation in another configuration example of the magnetic field sensor system according to the third embodiment.

FIG. 18 is a diagram showing a use state of a flexible device including a conventional magnetic field sensor system.

FIG. 19 is a diagram showing one example of a use state of the magnetic field sensor system according to the third embodiment.

FIG. 20 is a diagram showing another example of the use state of the magnetic field sensor system according to the third embodiment.

FIG. 21 is a diagram for illustrating the size of an antenna in the third embodiment.

FIG. 22 is a diagram for illustrating a position-detectable region which can be detected by an antenna.

FIG. 23 is a diagram showing some examples of an identifier for identifying a position-detectable region.

FIG. 24 is a perspective view for illustrating one example of a holder that rotatably holds an antenna.

FIG. 25 is a perspective view for illustrating another example of the holder that rotatably holds an antenna.

FIG. 26 is a diagram showing another example of the identifier.

FIG. 27 is a flowchart for illustrating one example of the operation of the magnetic field sensor system in the case of using an antenna including the identifier of FIG. 26.

FIG. 28 is a diagram showing an installation state of a test one-axis coil.

FIG. 29 is a flowchart for illustrating one example of the operation of the magnetic field sensor system in the case of using a test one-axis coil.

FIG. 30A is a diagram for illustrating the configuration of an antenna in a configuration example of a magnetic field sensor system according to a modification.

FIG. 30B is a diagram for illustrating a preferred arrangement of respective one-axis coils in FIG. 30A.

### DETAILED DESCRIPTION OF THE INVENTION

Hereinafter, embodiments for carrying out the present invention will be described with reference to the drawings.

### First Embodiment

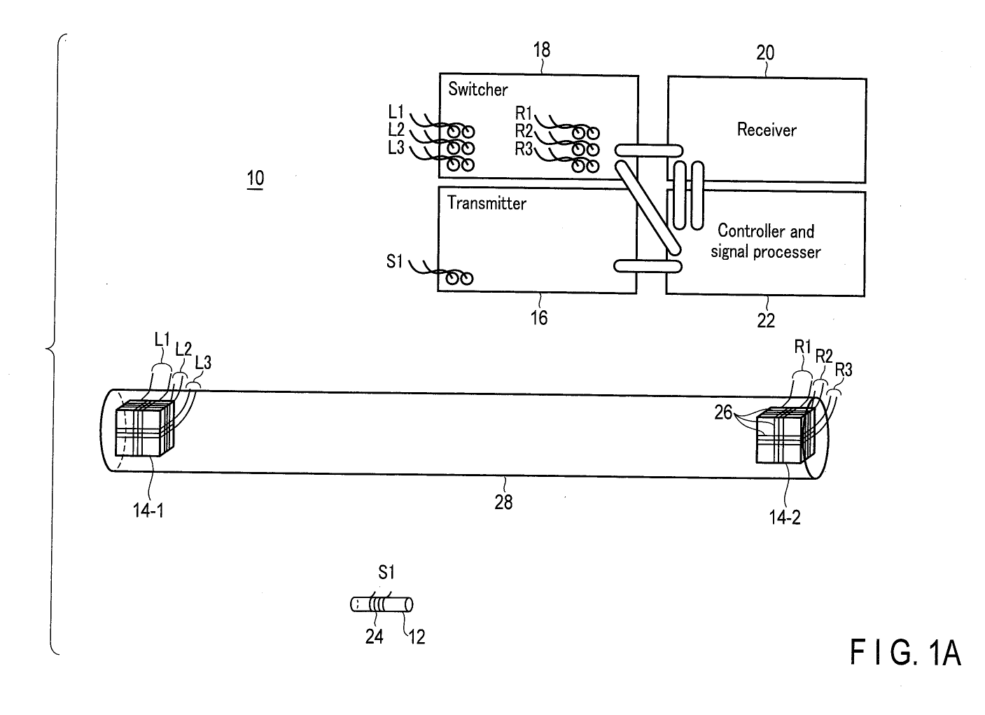

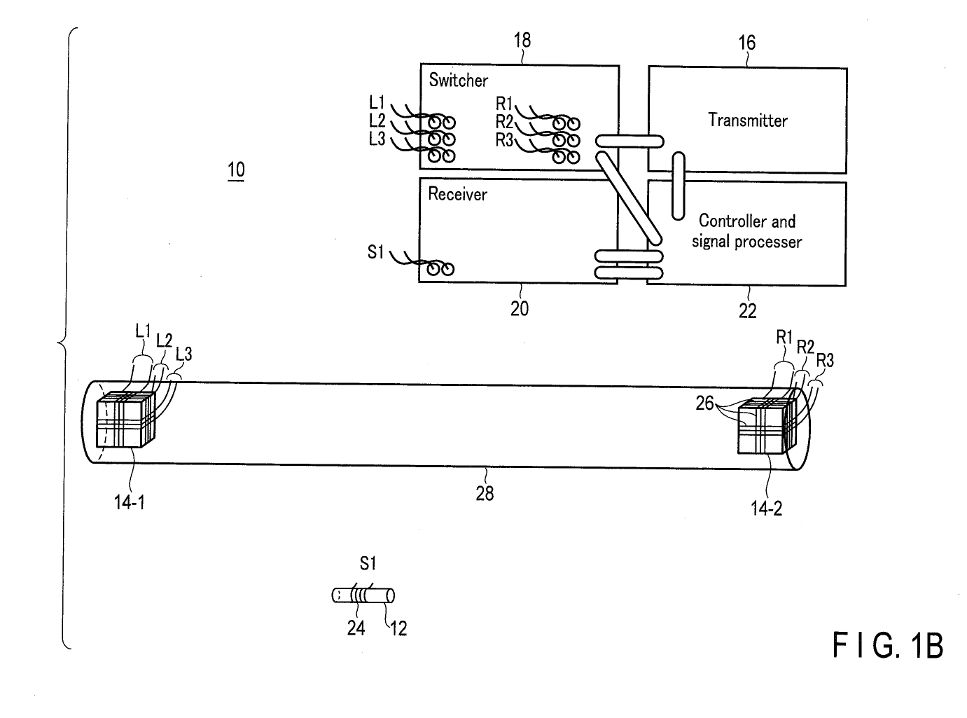

As shown in FIG. 1A, a magnetic field sensor system 10 according to a first embodiment of the present invention includes a one-axis coil 12, first and second three-axis coils 14-1 and 14-2, a transmitter 16, a switcher 18, a receiver 20, and a controller and signal processor 22. Here, in FIG. 1A, the wiring is partially omitted for the sake of clarity. Actually, the one-axis coil 12 is connected to the transmitter 16, and the first and second three-axis coils 14-1 and 14-2 are connected to the switcher 18.

The controller and signal processor 22 may include such an integrated circuit as a Central Processing Unit (CPU), an Application Specific Integrated Circuit (ASIC), or a Field Programmable Gate Array (FPGA). The controller and signal processor 22 may be one integrated circuit or the like, or may be a combination of a plurality of integrated circuits or the like. The operations of these integrated circuits are performed in accordance with a program recorded in a storage device or a storage area (not shown) provided inside or outside the controller and signal processor 22.

The transmitter 16 is connected to the controller and signal processor 22. The transmitter 16 may pass an electric current through a coil 24 of the one-axis coil 12, in accordance with a control signal from the controller and signal processor 22. The coil 24 is a one-axis magnetic field generation element. When the electric current is passed through the coil 24, the one-axis coil 12 may function as a one-axis magnetic field generator for generating a magnetic field. The one-axis coil 12 as a one-axis magnetic field generator is disposed in a detection target portion of an unillustrated flexible member.

Each of the first and second three-axis coils 14-1 and 14-2 is provided with the coils 26. Each coil 26 is a one-axis magnetic field detection element capable of detecting a magnetic field component in mutually linearly independent direction. The switcher 18 is connected to the receiver 20 and the controller and signal processor 22. According to a control signal from the controller and signal processor 22, the switcher 18 selectively connects one of the three coils 26 respectively provided in the first and second three-axis coils 14-1 and 14-2 to the receiver 20.

The receiver 20 is further connected to the controller and signal processor 22. According to the control signal from the controller and signal processor 22, the receiver 20 outputs a detected signal of the coil 26 input from the switcher 18 to the controller and signal processor 22. Therefore, each of the first and second three-axis coils 14-1 and 14-2 functions as a three-axis magnetic field detector for detecting magnetic fields.

The first and second three-axis coils 14-1 and 14-2 are arranged on a substantially straight line and are housed in a common bar-shaped exterior to constitute an antenna 28. Here, “a substantially straight line” means that part of the first three-axis coil 14-1 and part of the second three-axis coil 14-2 respectively exist on a certain straight line, not to say that the center of gravity of the first three-axis coil 14-1 and the center of gravity of the second three-axis coil 14-2 are aligned on the certain straight line. Furthermore, it also encompasses that part of the first three-axis coil 14-1 and part the first three-axis coil 14-1 may not exist on a certain straight line, as long as this does not give a large error to a detection result.

In the magnetic field sensor system 10 shown in FIG. 1A, the one-axis coil 12 serves as a transmitter of a magnetic field, and the three-axis coils 14-1 and 14-2 serve as magnetic field receivers. By reversing the role, as shown in FIG. 1B, the three-axis coils 14-1 and 14-2 may be used as transmitters and the one-axis coil 12 may be used as a receiver. This is because a following first magnetic field strength agrees with a following second magnetic field strength. The first magnetic field strength is a magnetic field strength when measuring a magnetic field strength of a specific one-axial direction component on the three-axis coils 14-1 or 14-2 in a case that the one-axis coil 12 sets as a transmitter of a magnetic dipole. The second case magnetic field strength is a magnetic field strength when measuring a magnetic field strength of a one-axial direction component on the one-axis coil 12 in a case that each one-axis of the three-axis coils 14-1 or 14-2 sets as a transmitter of a magnetic dipole.

Hereinafter, an example will be described in which the one-axis coil 12 is used as a one-axis magnetic field generator, i.e., a transmitter of a magnetic dipole.

The transmitter of the magnetic dipole may generate a DC dipole (for example, a DC current is passed through the coil 24) or an AC dipole (for example, an AC current is passed through the coil 24). For the AC dipole, for example, a coil 26 etc., which has sensitivity to its axis component is used for each axis of the receiver.

In the present embodiment, the coils 24 and 26 are used, but the coils are not necessarily used. For example, it is also possible to use a permanent magnet to generate a DC dipole. For the DC dipole, for example, a hall element, etc., which is sensitive to its axis component, can be used for each axis of the receiver.

Each of the three-axis coils 14-1 and 14-2, which are three-axis magnetic field detectors, is a group of coils capable of measuring three components in mutually linearly independent directions. FIGS. 1A and 1B illustrate, as examples, concentric three-axis coils 14-1 and 14-2. For example, hall elements used in the case of DC does not becomes completely concentric, but it is sufficient that a group of three components in mutually linearly independent directions can be measured thereby.

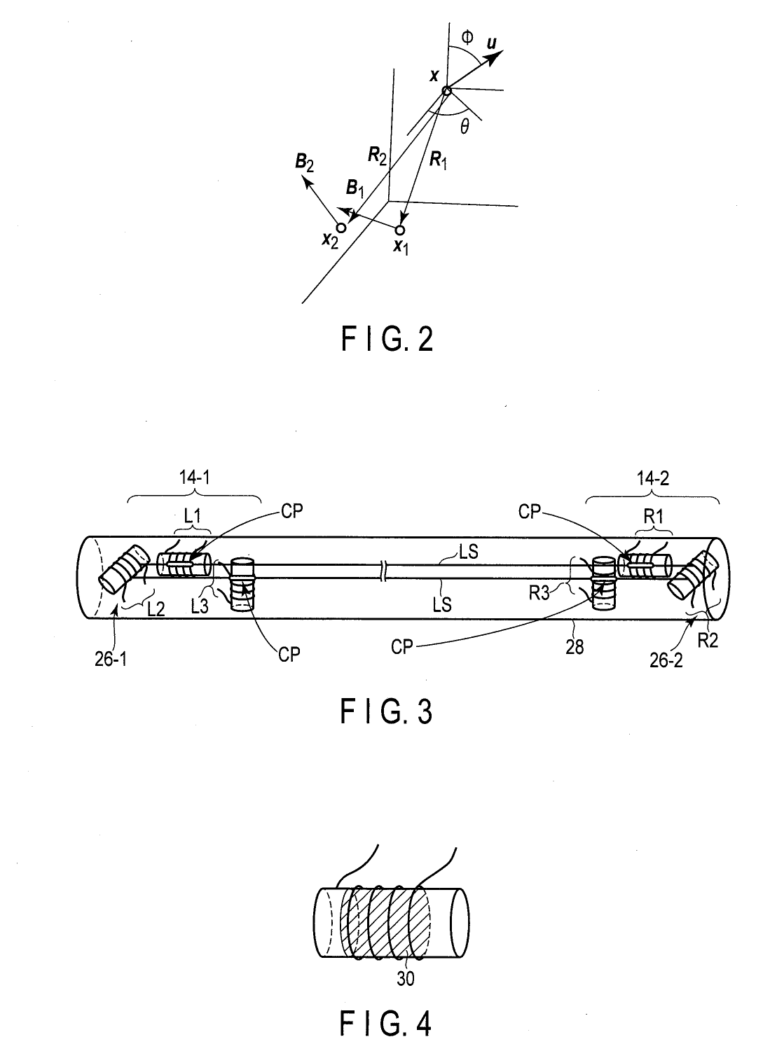

Here, the operation of the magnetic field sensor system 10 according to the first embodiment will be described with reference to FIG. 2. In the drawings and the specification, vectors are shown in bold italic letters.

When a magnetic dipole is generated from the one-axis coil 12, i.e., from a one-axis magnetic field generator x, in FIG. 2, six magnetic field signals B₁=(B₁ₓ, B_{1y}, B_{1z}) and B₂=(B₂ₓ, B_{2y}, B_{2z}) of components in respective positions and directions are obtained by two three-axis coils 14-1 and 14-2, i.e., two three-axis magnetic field detectors x₁ and x₂. On the other hand, five unknown numbers indicating the coordinates and the directions (x, y, z, θ, φ) of the one-axis magnetic field generator are tied with the detected magnetic field signals B₁, B₂ by the following relational expressions of the magnetic dipole.

That is, the relational expression for the magnetic field signal B₁ is as follows:

$${B}_{1} = \frac{2 {k}_{c} cos {α}_{1}}{{R}_{1}^{3}} {u}_{{R}_{1}} + \frac{{k}_{c} sin {α}_{1}}{{R}_{1}^{3}} {u}_{{α}_{1}}$$

With the proviso that

u_{R}_{₁}=R₁/R₁

u_{α}_{₁}=(u×u_{R}_{₁})×u_{R}_{₁}/sin α₁

cos α₁=u·u_{R}_{₁}

Here, “x” denotes an outer product, and “·” denotes an inner product. “u” denotes a direction of the coil 24 of the one-axis coil 12 which is a one-axis magnetic field generator, and “R₁” denotes a vector from the one-axis coil 12 to the three-axis magnetic field detector x₁. “u” and “R₁” are represented as follows:

u=(sin θ cos φ, sin θ sin φ, cos θ)

R₁=((x₁−x),(y₁−y),(z₁−z))

The same equations hold true for the magnetic field signal B₂.

Therefore, since the number of variables is five (x, y, z, θ, φ) for the total of six equations, these variables can be determined.

Then, the controller and signal processor 22 performs a position estimation by using, for example, the Gauss-Newton method, etc. That is, the controller and signal processor 22 minimizes the sum of squares S of a difference between the magnetic field signals B′₁ and B′₂ calculated by the above equations (Sum of squares of the difference=(B₁−B′₁)²+(B₂−B′₂)²) for the detected magnetic field signal B₁=(B₁ₓ, B_{1y}, B_{1z}), the magnetic field signal B₂=(B₂ₓ, B_{2y}, B_{2z}), and the set position and direction to thereby estimate a position and a direction of the one-axis coil 12. In the Gauss-Newton method, starting from an appropriate variable initial value, S is minimized by successively determining points where S is smaller. (Reference: Experimental Data Analysis by Least Squares Method, The University of Tokyo Press, p. 97)

If the coils of the three-axis magnetic field detector are not concentric, the coordinates are different for each linearly independent component, and thus R₁ in the above equation is different for each component. However, although the constants x₁, y₁, and z₁ are different for each component, the variables x, y, and z are common, and therefore, the total number of variables does not change. That is, in the case where the coils of the three-axis magnetic field detector are not concentric, each coil, i.e., individual magnetic field detection element measures one component (magnetic field in a uᵢ direction (i=1 to n)) of the magnetic field at different places Rᵢ. Therefore, in one magnetic field detection element i, the left side B (Rᵢ)·uᵢ of the following relational expression is detected as a magnetic field signal. The magnetic field signal detected as variables (x, y, z, θ, φ) has the following relational expressions:

$$B ( {R}_{i } ) \cdot {u}_{i} = \frac{2 {k}_{c} cos {α}_{i 0}}{{R}_{i 0}^{3}} {u}_{{R}_{i 0}} \cdot {u}_{i} + \frac{{k}_{c} sin {α}_{i 0}}{{R}_{i 0}^{3}} {u}_{{α}_{i 0}} \cdot {u}_{i}$$

With the proviso that

u_{R}_{ᵢ₀}=Rᵢ₀/Rᵢ₀

u_{α}_{ᵢ₀}=(u×u_{R}_{ᵢ₀})×u_{R}_{ᵢ₀}/sin αᵢ₀

cos α₁=u·u_{R}_{ᵢ₀}

Where “u” denotes the direction of the coil 24 of the one-axis coil 12, and “Rᵢ₀” denotes a vector from the one-axis coil 12 to the magnetic field detection element xᵢ. “u” and “Rᵢ₀” are represented as follows:

u=(sin θ cos φ, sin θ sin φ, cos θ)

Rᵢ₀=((xᵢ−x),(yᵢ−y),(zᵢ−z))

When there are two three-axis magnetic field detectors of which coils are not concentric, n=6, and thus, there are six relational expressions for five variables. Therefore, the controller and signal processor 22 can estimate a position and a direction of the one-axis coil 12, for example, by the above-described method.

As described above, the controller and signal processor 22 functions as a calculator that calculates at least one of a spatial position and a spatial direction of the one-axis coil 12, which is a one-axis magnetic field generator.

There are also applications where it is unnecessary to determine the direction. In that case, the controller and signal processor 22 may output only the position to a desired device. For example, the controller and signal processor 22 may display only the position to an unillustrated display device.

According to the configuration of the magnetic field sensor system 10 of the first embodiment as described above, the following advantages are obtained. For example, a transmitter to be arranged inside a flexible member such as an endoscope insertion section requires only the one-axis coil 12 which is a one-axis magnetic field generator. Therefore, the transmitter can be reduced in size and diameter. In addition, since receivers to be disposed outside the flexible member are receivers in which a plurality of three-axis coils 14-1 and 14-2, which are respectively three-axis magnetic field detectors, are one-dimensionally arranged, the antenna 28 can be configured in a bar shape. Therefore, the antenna 28 will not become an impediment to the operator.

This is the same also in the case where the transmitter and the receiver are reversed as shown in FIG. 1B. That is, for example, a receiver to be arranged inside a flexible member such as an endoscope insertion section requires only the one-axis coil 12 which is a one-axis magnetic field detector. Therefore, the receiver can be reduced in size and diameter. In addition, since transmitters to be disposed outside the flexible member are transmitters in which a plurality of three-axis coils 14-1 and 14-2, which are respectively three-axis magnetic field generators, are one-dimensionally arranged, the antenna 28 can be configured in a bar shape. Therefore, the antenna 28 will not become an impediment to the operator.

It is more preferred that the two three-axis coils 14-1 and 14-2 be arranged as follows.

As shown in FIG. 3, it is possible to select one coil (a first coil 26-1), which is one one-axis magnetic field detection element or one one-axis magnetic field generation element in the first three-axis coil 14-1, and one coil (a second coil 26-2), which is one one-axis magnetic field detection element or one one-axis magnetic field generation element in the second three-dimensional coil 14-2. There is certain line segments LS for each of the coils, connecting a first magnetic field detection/generation region (magnetic field detection/generation region of the first coil 26-1) and a second magnetic field detection/generation region (magnetic field detection/generation region of the second coil 26-2). Here, the “magnetic field detection/generation region” is, for example, in a coil, a region in the coil and the space enclosed by the coil in which a magnetic field is detected or generated. Therefore, as shown in FIG. 4, the “magnetic field detection/generation region” indicates a region 30 including a so-called coil around which a conductor is coaxially wound, and a space enclosed by the coil. The coils other than the first and second coils 26-1 and 26-2 are arranged in such a manner that their magnetic field detection/generation regions intersect with these line segments LS, namely, so as to have intersection portions CP.

Alternatively, there may be a certain line segment LS which is common to all the coils other than the first and second coils 26-1 and 26-2 and which connects the magnetic field detection/generation regions of the first coil 26-1 and the second coil 26-2. In this case, all the coils other than the first and second coils 26-1 and 26-2 may be arranged in such a manner that all the magnetic field detection/generation regions of the coils other than the first and second coils 26-1 and 26-2 intersect with the line segment LS.

In either method, the two three-axis coils 14-1 and 14-2 are arranged in a slim arrangement. Therefore, the antenna 28 can be formed in a thin rod shape, and the antenna 28 will not become an impediment to the operator.

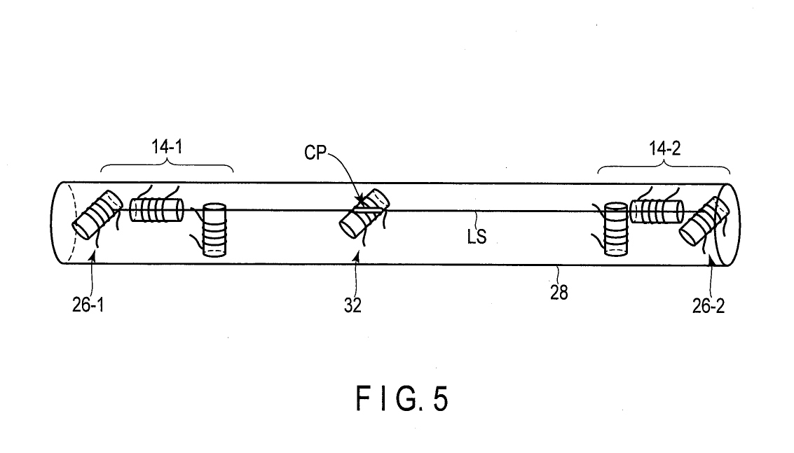

Further, as shown in FIG. 5, even in the case where an additional coil 32 is disposed, the additional coil 32 may be disposed similar to the coils other than the first and second coils 26-1 and 26-2. For example, there is a certain line segment LS for each of the coils, connecting the magnetic field detection/generation regions of the first coil 26-1 and the second coil 26-2. In this case, the additional coil 32 is disposed in such a manner that the magnetic field detection/generation region of the additional coil 32 intersects with this line segment LS, namely, so as to have an intersection portion CP. Alternatively, there is a certain line segment which is common to all the coils other than the first and second coils 26-1 and 26-2 and which connects the first coil 26-1 and the second coil 26-2. In this case, the additional coil 32 is disposed along with all the coils other than the first and second coils 26-1 and 26-2 in such a manner that the magnetic field detection/generation regions thereof intersect with the line segment LS. By adopting such an arrangement, the two three-axis coils 14-1 and 14-2 and the additional coil 32 are arranged in a slim arrangement.

Therefore, the antenna 28 can be formed in a thin rod shape and the antenna 28 will not become an impediment to the operator. The merit of disposing the additional coil 32 will be described in the second embodiment.

In magnetic field detection elements other than coils, for example, in a hall element, the volume of a semiconductor, in which a hall voltage is generated due to the influence of a magnetic field, is a detection region of a magnetic field corresponding to the magnetic field detection/generation region of the coil described above. Therefore, the above discussion holds true for the case of a hole element. Also, other magnetic field detection or generation elements require a volume for detection/generation for the detection/generation of a magnetic field, which can be referred to as a magnetic field detection/generation region. Then, the above discussion holds true for other magnetic field detection or generation elements.

The transmitter or the receiver to be arranged in a detection target portion of the flexible member is housed in a member with a small diameter. Therefore, it is preferable that the transmitter or the receiver is a one-axis magnetic field generator or a one-axis magnetic field detector.

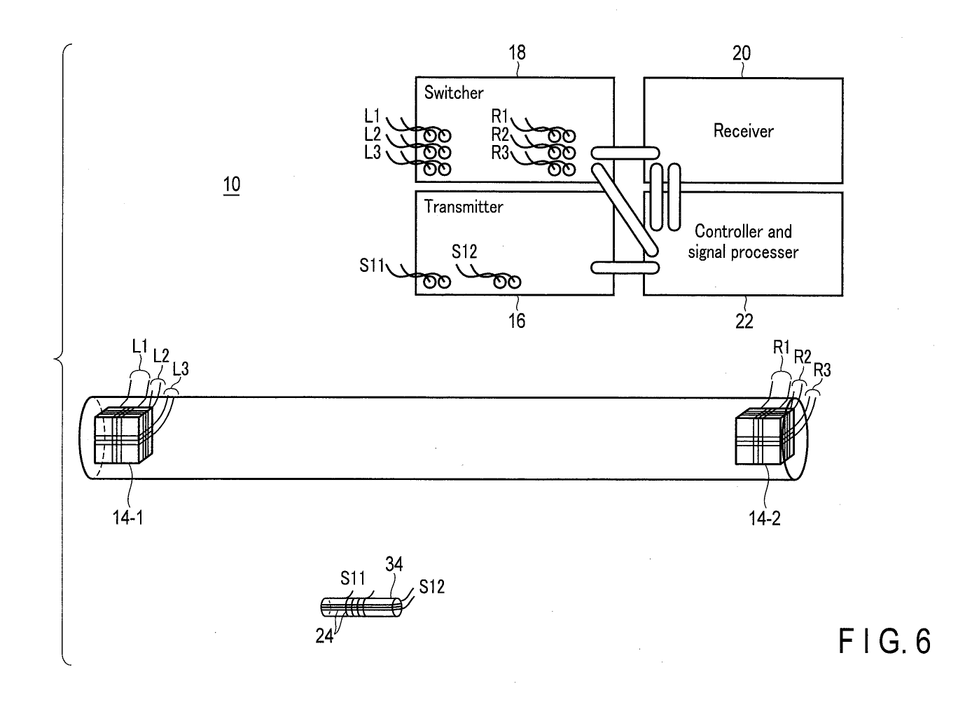

However, as shown in FIG. 6, for example, even in the case that the transmitter or the receiver is a two-axis magnetic field generator or a two-axis magnetic field detector such as a two-axis coil 34, it is possible to reduce the diameter thereof as compared with a three-axis magnetic field generator or detector.

When two three-axis coils 14-1 and 14-2, which are three-axis magnetic field detectors, are one-dimensionally arranged, there may be the case where the position and direction of the one-axis coil 12, which is a one-axis magnetic field generator, cannot be determined from two magnetic field signals B₁ and B₂ depending on the position and direction of the one-axis coil 12.

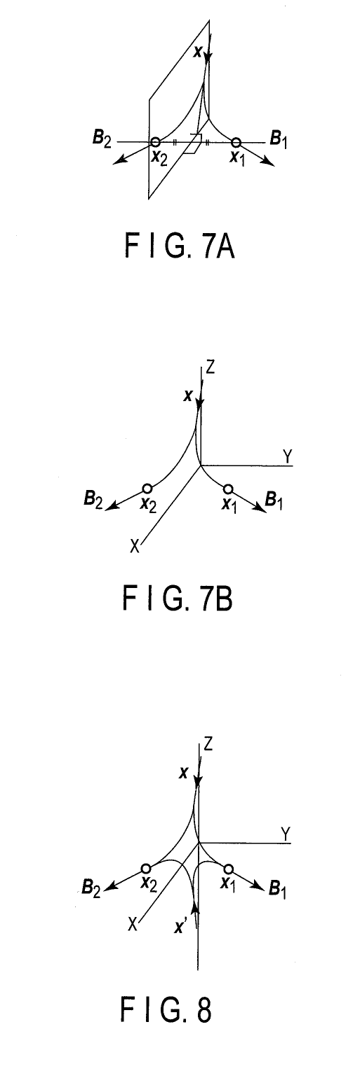

For example, as shown in FIG. 7A, a case is considered where a one-axis magnetic field generator is on the perpendicular bisecting plane of two three-axis magnetic field detectors and the direction of the one axis thereof is also in the perpendicular bisecting plane. In this case, since the magnetic field signals B₁, B₂ are symmetrical with respect to this perpendicular bisecting plane, the magnetic field signals B₁, B₂ are included in one plane that is orthogonal to this perpendicular bisecting plane and includes three-axis magnetic field detectors x₁, x₂. This plane is regarded as an XY plane. Furthermore, when a coordinate system is taken so that the position of the one-axis magnetic field generator is on a Z axis, FIG. 7A can be drawn as shown in FIG. 7B. Then, as shown in FIG. 8, by virtue of the symmetry, another one-axis magnetic field generator x′, which is symmetric with respect to the position of the one-axis magnetic field generator in the XY plane and whose direction is also symmetric with respect to this plane, also generates the same magnetic field signals B₁, B₂.

Therefore, in the case where the magnetic field signal B₁ detected by the three-axis coil 14-1 which is one of the three-axis magnetic field detectors and the magnetic field signal B₂ detected by the three-axis coil 14-2 which is the other three axis magnetic field detector have approximately such symmetry, the controller and signal processor 22, which is a calculator, obtains two solutions as a spatial position and/or a spatial direction of the one-axis coil 12 which is a one-axis magnetic field generator.

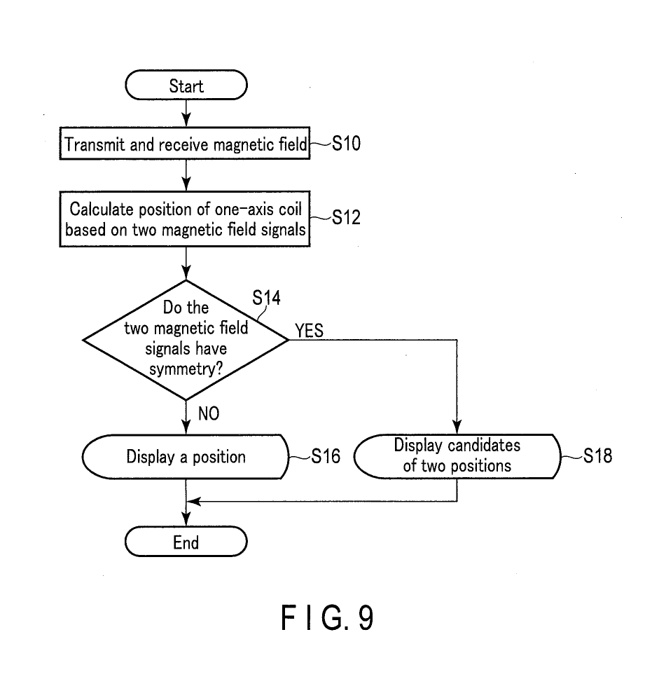

Therefore, in the magnetic field sensor system 10 according to the first embodiment, the controller and signal processor 22 operates as shown in FIG. 9. The controller and signal processor 22 firstly transmits and receives a magnetic field (step S10). That is, the controller and signal processor 22 causes the one-axis coil 12, which is a one-axis magnetic field generator, to generate a magnetic field and causes the two three-axis coils 14-1 and 14-2 which are two three-axis magnetic field detectors, to detect the magnetic field. Then, the controller and signal processor 22 calculates a spatial position and/or a spatial direction of the one-axis coil 12 based on the two magnetic field signals B₁, B₂ detected by the two three-axis coils 14-1 and 14-2 (step S12).

Thereafter, the controller and signal processor 22 determines whether or not two solutions are obtained as the spatial position and/or spatial direction of the one-axis coil 12, namely, whether or not the detected two magnetic field signals B₁ and B₂ have symmetry (step S14). In this step, when the controller and signal processor 22 has determined that the detected two magnetic field signals B₁, B₂ do not have symmetry, the controller and signal processor 22 outputs the calculated spatial position/or spatial direction of the one-axis coil 12 to the outside by, for example, displaying the calculation result on an unillustrated display device (step S16). In contrast, when the controller and signal processor 22 has determined that the detected two magnetic field signals B₁, B₂ have symmetry, the controller and signal processor 22 outputs, as candidates for the position and/or direction, two positions and/or two directions obtained by the calculation to the outside by, for example, displaying the candidates on the unillustrated display device (step S18).

As described above, when the detected magnetic field signals B₁ and B₂ have approximate symmetry, the controller and signal processor 22 presents two positions and/or two directions as candidates.

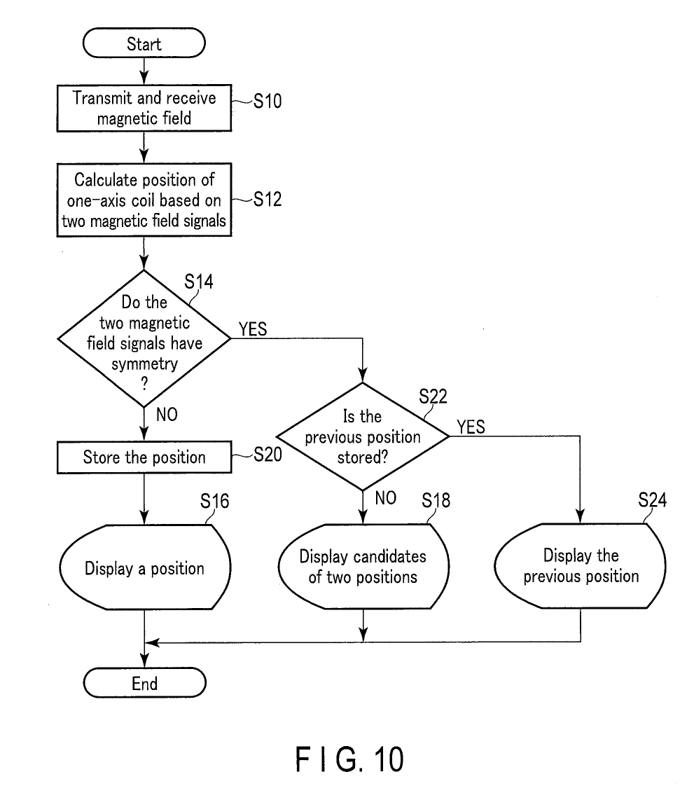

Alternatively, the controller and signal processor 22 may operate as shown in FIG. 10. When the controller and signal processor 22 has determined that the detected two magnetic field signals B₁, B₂ do not have symmetry, the controller and signal processor 22 stores the calculated spatial position and/or spatial direction of the one-axis coil 12 in an unillustrated internal memory, etc. (step S20). When the controller and signal processor 22 has determined that the detected two magnetic field signals B₁, B₂ have symmetry, the controller and signal processor 22 further confirms whether or not the previous position and/or direction, which are the previous calculation results, are stored in the unillustrated internal memory (step S22). Then, if the previous position and/or direction are not stored in the unillustrated internal memory, the controller and signal processor 22 proceeds to the operation of the step S18. On the other hand, if it is determined that the previous position and/or direction are stored in the unillustrated internal memory, the controller and signal processor 22 outputs the stored previous position/or direction, as a position and/or direction calculated this time, to the outside by, for example, displaying the position and/or direction calculated this time on the unillustrated display device (Step S24).

As described above, when a spatial position and/or a spatial direction are detected in time series, and when the detected magnetic field signals B₁, B₂ have approximate symmetry, the calculation result at the immediately preceding detection may be presented.

The magnetic field sensor system according to the first embodiment as described above includes a one-axis coil 12, three axis coils 14-1 and 14-2 (and a one-axis coil 32), and a controller and signal processor 22. The one-axis coil 12 is a one-axis magnetic field generator or detector for generating or detecting a magnetic field. The three axis coils 14-1 and 14-2 (and a one-axis coil 32) are a plurality of magnetic field detectors or generators for detecting or generating a magnetic field. The controller and signal processor 22 is a calculator that calculates at least one of a spatial position and a spatial direction of the one-axis magnetic field generator or detector from the detection result of the magnetic field. The plurality of magnetic field detectors or generators are arranged on a substantially straight line.

Alternatively, the magnetic field sensor system according to the first embodiment includes a two-axis coil 34, three axis coils 14-1 and 14-2 (and a one-axis coil 32), and a controller and signal processor 22. The two-axis coil 34 is a two-axis magnetic field generator or detector for generating or detecting a magnetic field for each axis. The three-axis coils 14-1 and 14-2 (and the one-axis coil 32) are a plurality of magnetic field detectors or generators for detecting or generating a magnetic field. The controller and signal processor 22 is a calculator that calculates at least one of a spatial position and a spatial direction of the two-axis magnetic field generator or detector from the detection result of the magnetic field. The plurality of magnetic field detectors or generators are arranged on a substantially straight line.

In this manner, as a result of arranging a plurality of magnetic field detectors or generators on a substantially straight line rather than on a plane, it is possible to detect the position of a detection target portion in a flexible member with a small diameter without preventing the movement of the operator outside the flexible member as much as possible.

Each of the plurality of magnetic field detectors or generators is a three-axis coil 14-1 or 14-2 which is a three-axis magnetic field detector or generator that detects or generates a magnetic field for each axis, and the plurality of three-axis magnetic field detectors or generators are arranged on a substantially straight line.

Alternatively, the plurality of magnetic field detectors or generators include a plurality of three-axis magnetic field detectors or generators that detect or generate a magnetic field for each axis, and the three-axis coils 14-1, 14-2 which are all the magnetic detectors or generators including three-axis coils which are the plurality of magnetic field detectors or generators and the one-axis coil 32 are arranged on a substantially straight line.

As a result of adopting such a configuration, the three-axis coils 14-1 and 14-2 are arranged in a slim arrangement. Therefore, the antenna 28 can be formed in a thin rod shape, and will not become an impediment to the operator.

In the case where the magnetic fields detected by using the plurality of three-axis magnetic field detectors or generators have symmetry, the calculator uses at least one of the spatial position and the spatial direction of the one-axis or two-axis magnetic field generator or detector calculated one time ago as at least one of a current position and a current direction.

As described above, when a position and/or a direction is detected in time series and when the detected magnetic field signals have approximate symmetry, the calculation result at the immediately preceding detection is used, making it possible to reduce the possibility of presenting an erroneous measurement result to the operator.

Alternatively, in the case where the magnetic fields detected by using the plurality of three-axis magnetic field detectors or generators have symmetry, the calculator may be configured to calculate a plurality of candidates of at least one of the spatial positions and directions of the one-axis or two-axis magnetic field generator or detector.

As described above, when the detected magnetic field signals have approximate symmetry, it is possible to present to the operator two measurement results by obtaining two positions and/or two directions as candidates to allow the operator to make a judgement of the at least one of the spatial positions and directions of the one-axis or two-axis magnetic field generator or detector.

### Second Embodiment

Next, a magnetic field sensor system 10 according to a second embodiment of the present invention will be described.

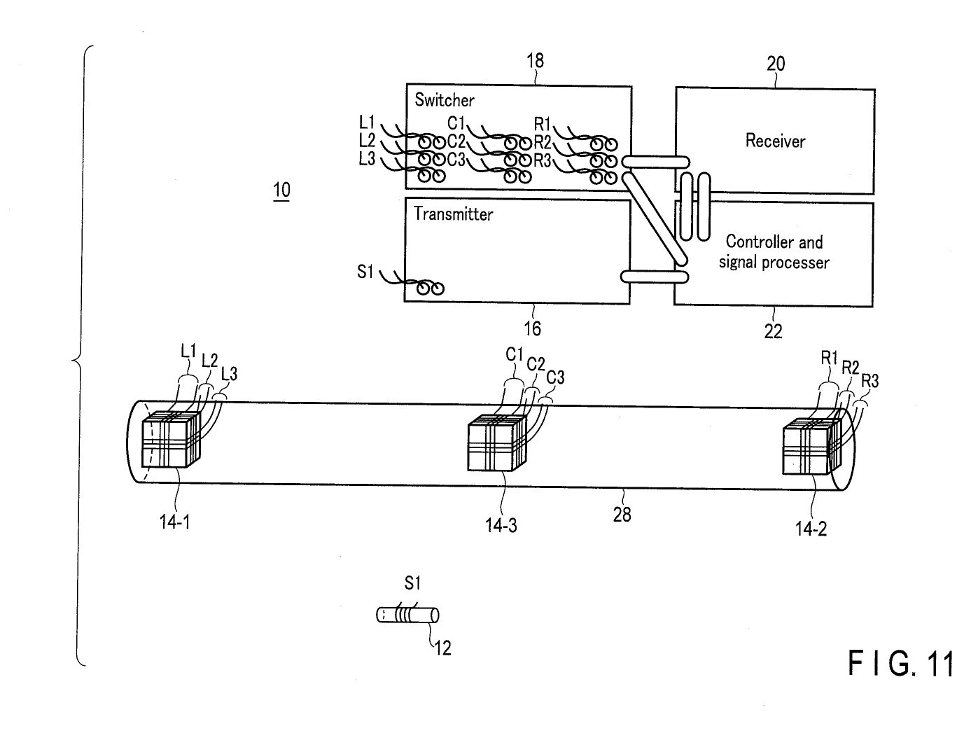

As shown in FIG. 11, the magnetic field sensor system 10 according to the second embodiment houses, in the exterior of the antenna 28, in addition to the two first and second three-axis coils 14-1 and 14-2 which are respectively three-axis magnetic field detectors, a third three-axis coil 14-3 which is a similar three-axis magnetic field detector. The first to third three-axis coils 14-1, 14-2, and 14-3 are arranged on a substantially straight line. In accordance with a control signal from a controller and signal processor 22, a switcher 18 selectively connects one of coils 26, which are three one-axis magnetic field detection elements respectively provided in the first to third three-axis coils 14-1, 14-2, and 14-3, to a receiver 20.

As described in the first embodiment, when a magnetic field signal detected by the first three-axis coil 14-1 and a magnetic field signal detected by the second three-axis coil 14-2 have approximate symmetry, two solutions are always obtained as a spatial position and/or direction of the one-axis coil 12. Therefore, in the second embodiment, this problem is solved by increasing one or more three-axis magnetic field detectors without changing the arrangement of the magnetic field detectors in which the antenna 28 has a one-dimensional rod shape. That is, even when a magnetic field signal detected by the first three-axis coil 14-1 and a magnetic field signal detected by the second three-axis coil 14-2 have symmetry, for example, a pair of the first three-axis coil 14-1 and the third three-axis coil 14-3 has no symmetry. Therefore, the controller and signal processor 22 can uniquely calculate a position and/or a direction of the one-axis coil 12 based on the magnetic field signal detected by the first three-axis coil 14-1 and the magnetic field signal detected by the third three-axis coil 14-3.

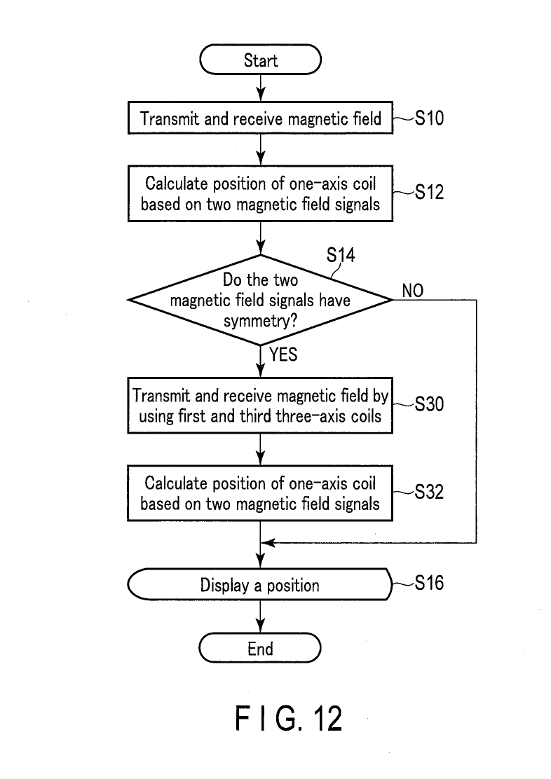

Therefore, the magnetic field sensor system 10 according to the second embodiment operates as shown in FIG. 12. That is, similarly to the first embodiment, the controller and signal processor 22 transmits and receives a magnetic field by using the one-axis coil 12 and the first and second three-axis coils 14-1 and 14-2 (Step S10). Then, the controller and signal processor 22 calculates a spatial position and/or a spatial direction of the one-axis coil 12 from two magnetic field signals detected by the two three-axis coils 14-1 and 14-2 (Step S12). Thereafter, the controller and signal processor 22 determines whether or not the detected two magnetic field signals have symmetry (step S14). When the controller and signal processor 22 has determined that the two magnetic field signals do not have symmetry, the controller and signal processor 22 outputs the calculated spatial position and/or direction of the one-axis coil 12 to the outside by, for example, displaying the calculation results on an unillustrated display device (step S16).

In contrast, when the controller and signal processor 22 has determined that the detected two magnetic field signals have symmetry, the controller and signal processor 22 changes one of three-axis magnetic field detectors to be used from the second three-axis coil 14-2 to the third three-axis coil 14-3 and transmits and receives a magnetic field (step S30). Thereafter, the controller and signal processor 22 calculates a spatial position and/or a spatial direction of the one-axis coil 12 from the two magnetic field signals detected by the two three-axis coils 14-1 and 14-3 (Step S32). Then, the controller and signal processor 22 outputs the obtained spatial position and/or spatial direction of the one-axis coil 12 to the outside by, for example, displaying the obtained result on an unillustrated display device (step S16).

Also, instead of using three or more three-axis magnetic field detectors in pairs as described above, three or more sets of three or more three-axis magnetic field detectors may be used to increase the number of detection points of a magnetic field and to improve the accuracy of the calculation of the position and/or the direction. For the purpose of increasing the number of detection points of a magnetic field and improving the accuracy of the position and/or direction calculation, it is not always necessary to increase the number of three-axis magnetic field detectors, and one or more additional coils 32 which are one-axis magnetic field detectors may be added as shown in FIG. 5.

Alternatively, the following configuration may be used. That is, when the one-axis coil 12, which is a one-axis magnetic field generator, is close to the first three-axis coil 14-1 disposed at one end of the antenna 28, two or more magnetic field signals are detected by using two or more nearby three-axis coils to calculate the position and/or direction of the one-axis coil 12. When the one-axis coil 12, which is a one-axis magnetic field generator, is close to the second three-axis coil 14-2 disposed at the other end of the antenna 28, i.e., disposed on the opposite side, two or more magnetic signals are detected by using two or more nearby three-axis coils to calculate the position and/or direction of the one-axis coil 12. According to such usage of three or more three-axis coils, it is possible to calculate the position and/or direction with less error.

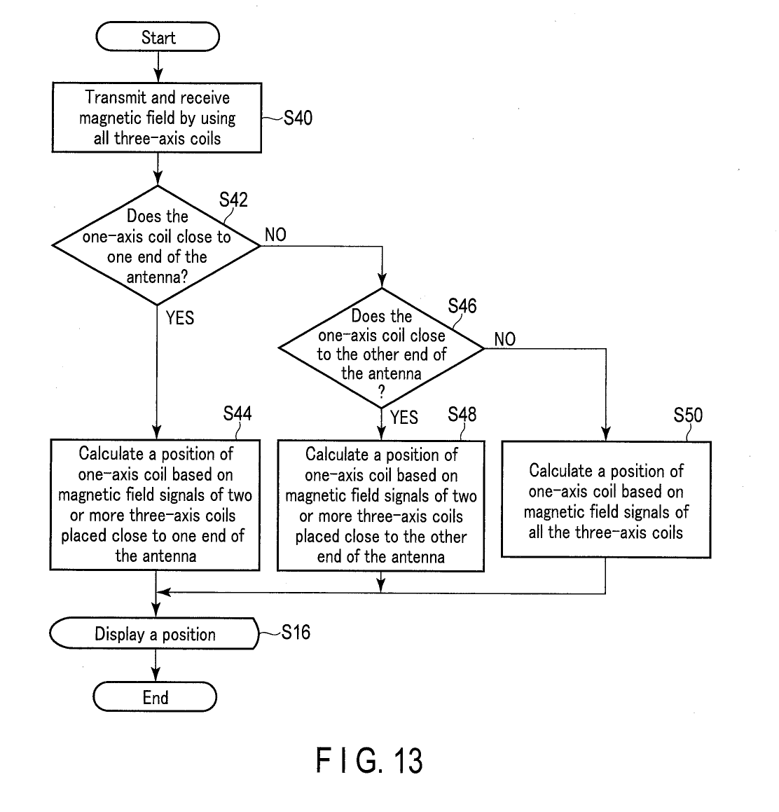

In the case of such usage of three-axis coils, the magnetic field sensor system 10 according to the second embodiment operates as shown in FIG. 13.

That is, all three or more three-axis coils, for example, three three-axis coils 14-1, 14-2, and 14-3, which are respectively three-axis magnetic field detectors, are used to receive a magnetic field (step S40). Then, the controller and signal processor 22 determines whether or not the one-axis coil 12 as a one-axis magnetic generator is close to a three-axis coil disposed at one end of the antenna 28, for example, the first three-axis coil 14-1, from the respective magnetic field signals (step S42).

When it is determined in step S42 that the one-axis coil 12 is close to the first three-axis coil 14-1, the controller and signal processor 22 performs the following operation. That is, the controller and signal processor 22 calculates a spatial position and/or a spatial direction of the one-axis coil 12, for example, from two magnetic field signals detected by two or more three-axis coils close to one end of the antenna 28, for example, two three-axis coils 14-1 and 14-3 (step S44). Then, the controller and signal processor 22 outputs the obtained spatial position and/or spatial direction of the one-axis coil 12 to the outside by, for example, displaying the obtained result on an unillustrated display device (step S16).

In contrast, when the controller and signal processor 22 has determined in step S42 that the one-axis coil 12 is not close to the first three-axis coil 14-1, it performs the following operation. That is, the controller and signal processor 22 determines whether or not the one-axis coil 12, which is a one-axis magnetic field generator, is close to a three-axis coil disposed at the other end of the antenna 28, for example, the second three-axis coil 14-2, from each of the magnetic field signals obtained in step S40 (step S46).

When the controller and signal processor 22 determines in the step S46 that the one-axis coil 12 is close to the second three-axis coil 14-2, it performs the following operation. That is, the controller and signal processor 22 calculates the spatial position and/or spatial direction of the one-axis coil 12 from two or more three-axis coils close to the other end of the antenna 28, for example, two three-axis coils 14-2 and 14-3 (step S48). Then, the controller and signal processor 22 outputs the obtained spatial position and/or spatial direction of the one-axis coil 12 to the outside by, for example, displaying the obtained result on an unillustrated display device (step S16).

When the controller and signal processor 22 determines in the step S46 that the one-axis coil 12 is not close to the third three-axis coil 14-3, the controller and signal processor 22 performs the following operation. That is, the controller and signal processor 22 calculates the spatial position and/or spatial direction of the one-axis coil 12 from all the magnetic field signals obtained in step S40 (step S50). Then, the controller and signal processor 22 outputs the obtained spatial position and/or direction of the one-axis coil 12 to the outside by, for example, displaying the obtained result on an unillustrated display device (step S16).

Also in the present embodiment, as a matter of course, the roles of the one-axis coil 12 and the three-axis coils 14-1, 14-2, 14-3 are reversed so that the one-axis coil 12 may be used as a magnetic field detector, and the three-axis coils 14-1, 14-2, and 14-3 may be used as magnetic field generators, similarly to the first embodiment.

Furthermore, the fact that the two-axis coil 34 may be used instead of the one-axis coil 12 is the same as in the first embodiment.

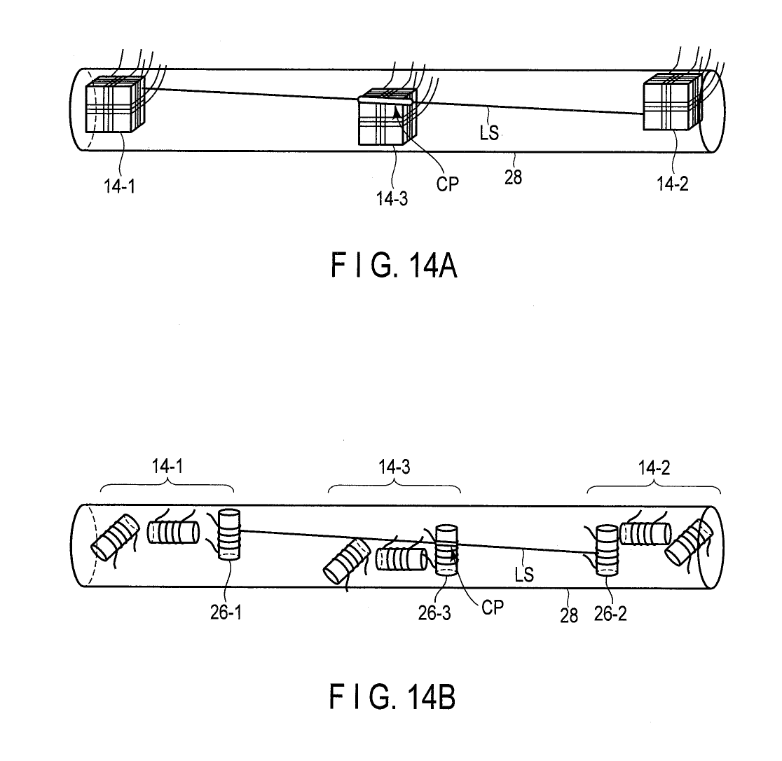

In addition, as shown in FIG. 14A, the added third three-axis coil 14-3 is disposed in such a manner that a line segment LS connecting the magnetic field detection/generation region of the first three-axis coil 14-1 and the magnetic field detection/generation region of the second three-axis coil 14-2 intersects with the magnetic field detection/generation region of this third three-axis coil 14-3, namely, so as to have an intersection portion CP. By arranging the coils under this condition, the exterior of the antenna 28 does not change much as compared with that of the first embodiment despite the addition of the third three-axis coil 14-3, and a slim outline that does not interfere with the operation of the operator is maintained.

Furthermore, as shown in FIG. 14B, for coils which are respective one-axis magnetic field detection/generation elements of the third three-axis coil 14-3, there is a certain line segment LS connecting the magnetic field detection/generation regions of corresponding coils of the first three-axis coil 14-1 and the second three-axis coil 14-2. For example, there is a line segment LS that connects a certain magnetic field detection/generation region of a coil 26-1 and a magnetic field generation/detection region of a corresponding coil 26-2 of the third three-axis coil 14-2. It is even better to arrange each of the three-axis coils 14-1 to 14-3 so that this line segment LS and a magnetic field detection/generation region of a corresponding coil 26-3 of the third three-axis coil 14-3 intersect with each other.

As described above, the magnetic field sensor system according to the second embodiment includes three or more three-axis coils 14-1, 14-2, and 14-3 as a plurality of three-axis magnetic field detectors or generators.

This solves the problem of two solutions appearing when the magnetic fields have symmetry in two three-axis magnetic field detectors or generators.

That is, in the second embodiment, the one-axis or two-axis magnetic field generator or the one-axis or two-axis detector is a one-axis or two-axis magnetic field generator, and the three or more three-axis magnetic field detectors or three or more three-axis magnetic field generators are three or more three-axis magnetic field detectors. In this case, the calculator selects two or more three-axis magnetic field detectors from among the three or more three-axis magnetic field detectors, based on a preset judgement standard regarding values of magnetic fields measured by two or more three-axis magnetic field detectors. Then, the calculator calculates at least one of a spatial position and a spatial direction of the one-axis or two-axis magnetic field generator based on the detection results of the selected two or more three-axis magnetic field detectors.

Alternatively, the one-axis or two-axis magnetic field generator or the one-axis or two-axis detector is a one-axis or two-axis magnetic field detector, and the three or more three-axis magnetic field detectors or three or more three-axis magnetic field generators are three or more three-axis magnetic field generators. In this case, the calculator selects two or more three-axis magnetic field generators from among the three or more three-axis magnetic field generators, based on a preset judgement standard regarding values of magnetic fields, which each of the values of the magnetic fields is generated for each axis by the two or more three-axis magnetic field generators and measured by the one-axis or two-axis magnetic field detector. Then, the calculator calculates at least one of a spatial position and a spatial direction of the one-axis or two-axis magnetic field detector, from the detection results of the one-axis or two-axis magnetic detector for the magnetic field individually generated for each axis by the selected two or more three-axis magnetic field generators.

In this way, it is possible to calculate at least one of a spatial position and a spatial direction of a one-axis or two-axis magnetic field generator or detector by selecting two three-axis magnetic field detectors or generators for use in calculation of the position and/or direction based on a judgement standard.

The preset judgement standard is a preset judgement standard related to the symmetry of magnetic fields, the preset judgement standard regarding to the values of the magnetic fields measured by the two or more three-axis magnetic field detectors.

Alternatively, the preset judgement standard is a preset judgement standard related to the symmetry of magnetic fields, the preset judgement standard being for values of the magnetic fields when individually generating for each axis by the two or more three axis magnetic field generators and measuring by the one-axis magnetic field detector.

The preset judgment standard may be a preset judgement standard related to candidate positions estimated from values of magnetic fields, the preset judgement standard being with respect to the values of the magnetic fields measured by the two or more three-axis magnetic field detectors.

Alternatively, the preset judgment standard may be a preset judgement standard related to candidate positions estimated from values of magnetic fields, the preset judgement standard being with respect to the values of the magnetic fields when generating individually for each axis by the two or more three-axis magnetic field generators and measuring by the one-axis magnetic field detector.

The judgment standard is stored in an unillustrated internal memory, etc., of the controller and signal processor 22.

In the case where the magnetic fields detected using the selected two three-axis magnetic field detectors or generators have symmetry, another combination of the three-axis magnetic field detectors or three-axis magnetic field generators is selected to detect at least one of a spatial position and a spatial direction of the one-axis or two-axis magnetic field generator or detector.

In this way, erroneous measurement can be reduced by using two sets of three or more three-axis magnetic field detectors or generators at a time.

In the case where the one-axis or two-axis magnetic field generator or detector is close to the three-axis magnetic field detector or generator at one end among the three or more three-axis magnetic field generators or generators, the calculator may calculate at least one of a spatial position and a spatial direction of the one-axis or two-axis magnetic field detector or generator, based on detection results using two or more three-axis magnetic field detectors or generators close to the three-axis magnetic field detector or generator at one end. Alternatively, in the case where the one-axis or two-axis magnetic field generator or detector is close to the three-axis magnetic field detector or generator at the other end among the three or more three-axis magnetic field generators or generators, the calculator may calculate at least one of the spatial position and the spatial direction of the one-axis or two-axis magnetic field detector or generator, based on detection results using two or more three-axis magnetic field detectors or generators close to the three-axis magnetic field detector or generator at the other end.

According to the use of three or more three-axis magnetic field detectors or generators as described above, it is possible to calculate a position and/or a direction with less error.

In addition, it is desired that the three-axis magnetic field detectors or three-axis magnetic field generators other than the first and second three-axis magnetic field detectors or generators which are two of the three or more three-axis magnetic field detectors or generators, be arranged in such a manner that respective magnetic field detection/generation regions thereof intersect with a line segment connecting the magnetic field detection/generation region of the first three-axis magnetic field detector or generator and the magnetic field detection/generation region of the second three-axis magnetic field detector or generator.

Alternatively, it is desired that the magnetic field detectors or generators other than the first and second magnetic field detectors or generators which are two of the plurality of magnetic field detectors or generators be arranged in such a manner that the respective magnetic field detection/generation regions thereof intersect with a line segment connecting the magnetic field detection/generation region of the first magnetic field detector or generator and the magnetic field detection/generation region of the second magnetic field detector or generator.

Furthermore, it is further desired that among the plurality of three-axis magnetic field detectors or generators, the magnetic field detection or generation elements for each axis other than the one-axis magnetic field detection or generation element in the first three-axis magnetic field detector or generator and the one-axis magnetic field detection or generation element in the second three-axis magnetic field detector or generator are arranged in such a manner that the respective magnetic field detection/generation regions intersect with a line segment connecting the magnetic field detection/generation region of the one-axis magnetic field detection or generation element of the first three-axis magnetic field detector or generator and the magnetic field detection/generation region of the one-axis magnetic field detection or generation element of the second three-axis magnetic field detector or generator.

With such a configuration, the exterior of the antenna 28 does not change much as compared with that of the first embodiment despite the addition of three-axis magnetic field detectors or generators other than the first and second three-axis magnetic field detectors or generators, and a slim outline that does not interfere with the operation of the operator is maintained.

### Third Embodiment

Next, a magnetic field sensor system 10 according to a third embodiment of the present invention will be described.

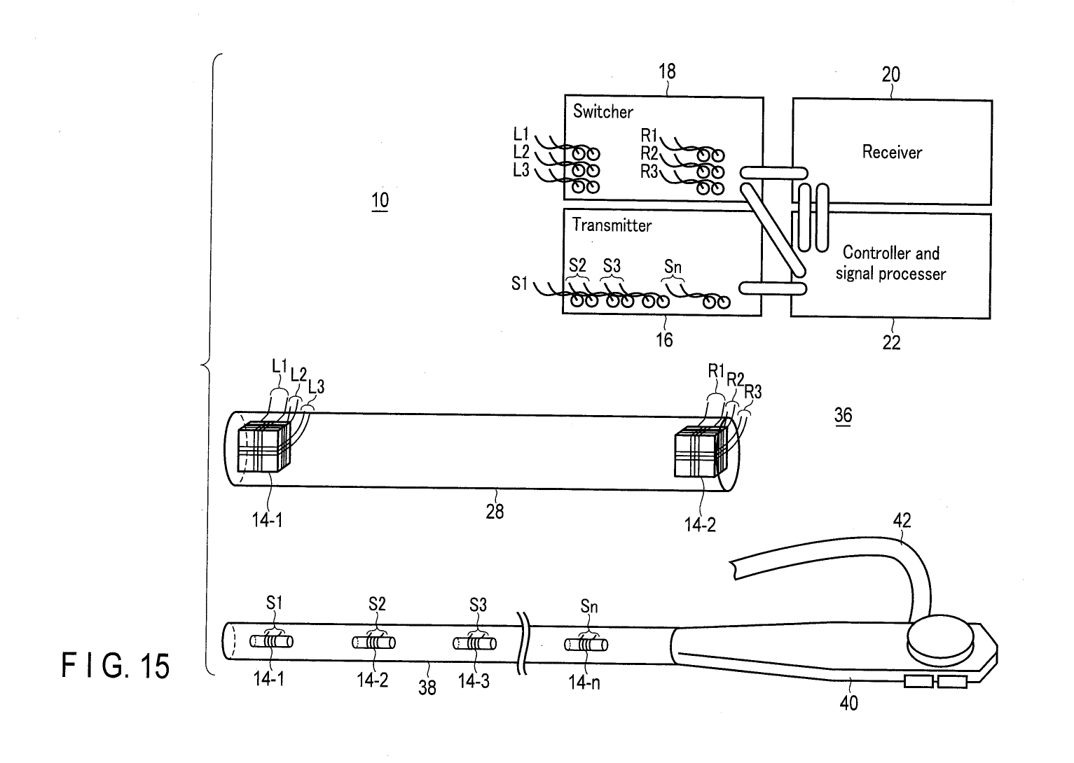

This embodiment is an example applied to an endoscope 36 as a flexible device. In the present embodiment, as shown in FIG. 15, the endoscope 36 includes an insertion portion 38 which is an example of a flexible member, an operation portion 40, and a cable 42. The insertion portion 38 is a flexible member that is inserted into a tubular object to be inspected such as an intestinal tract. The operation section 40 is connected to the proximal end of the insertion section 38 and gripped by an operator such as a doctor. The cable 42 connects the operation unit 40 and an unillustrated main body on which a light source device and an image processing device are mounted. Although not specifically illustrated, an endoscopic image processed by the image processing device of the main body is displayed on an unillustrated display device.

The magnetic field sensor system 10 according to the present embodiment has n pieces of one-axis coils 12-1, 12-2, 12-3, . . . , 12-n. These one-axis coils are arranged inside the insertion portion 38 which is a flexible member with a small diameter correspondingly to detection target portions arranged in the longitudinal direction of the insertion portion 38. These n pieces of one-axis coils 12-1, 12-2, 12-3, . . . , 12-n are connected to the transmitter 16 and each function as a one-axis magnetic field generator.

The transmitter 16, the switcher 18, the receiver 20, the controller and signal processor 22 may be incorporated into the main body of the endoscope 36, or may be arranged in a housing separate from the main body.

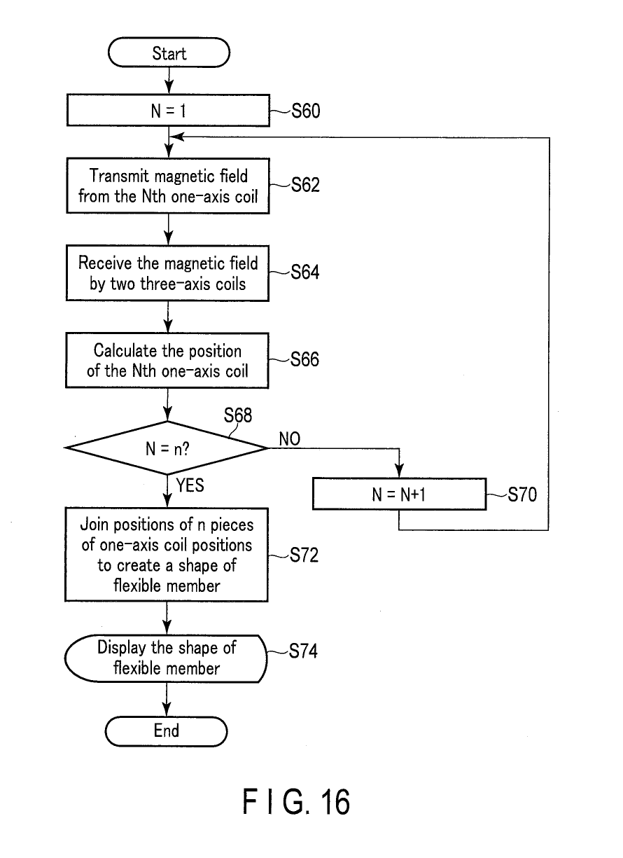

The magnetic field sensor system 10 having such a configuration operates as shown in the flowchart of FIG. 16. That is, the controller and signal processor 22 firstly initializes a variable counter N, which is internally constructed, to 1 (step S60).

Thereafter, the controller and signal processor 22 generates a magnetic field from the Nth one-axis coil among the n pieces of one-axis coils 12-1 to 12-n arranged inside the insertion section 38 of the endoscope 36, namely, causes the Nth one-axis coil to transmit a magnetic field (step S62). Then, the controller and signal processor 22 causes each of the two three-axis coils 14-1 and 14-2 in the antenna 28 to detect a magnetic field, namely, causes the two three-axis coils to receive the magnetic field (step S64). Then, the controller and signal processor 22 calculates a spatial position and/or a spatial direction of the Nth one-axis coil 12 from two magnetic field signals B₁ and B₂ detected by the two three-axis coils 14-1 and 14-2, and stores the calculation result in an unillustrated internal memory, etc. (step S66).

Next, the controller and signal processor 22 determines whether or not the value of the variable counter N is n (step S68). In this step, if it is determined that the value of the variable counter N is not yet n, the controller and signal processor 22 increments the value of the variable counter N by 1 (step S70) and repeats the processing from step S62. In this example, the value of the variable counter N is incremented by 1 from 1 to n, and processing is performed for all of the n pieces of one-axis coils 12-1 to 12-n, but conversely, it is a matter of course that the initial value of the variable counter N may be set to n, and the value of the variable counter N may be reduced from n to 1 one by one.

In this manner, a magnetic field is transmitted and received using each of the n pieces of one-axis coils 12-1 to 12-n to calculate a spatial position and/or a special direction for each of the n pieces of the one-axis coils 12-1 to 12-n.

If it is determined in step S68 that the value of the variable counter N is n, the controller and signal processor 22 joins spatial positions and/or spatial directions of the n pieces of the one-axis coils 12-1 to 12-n stored in the internal memory to create shape information of the insertion section 38 (step S72). The shape information of the insertion section 38 is displayed on a display device that displays an endoscopic image or another display device (step S74).

As described above, a plurality of one-axis coils 12 are installed in the insertion portion 38 of the endoscope 36, and the controller and signal processor 22 causes the respective one-axis coils 12 to transmit a magnetic field in a time-sequential order, and cause the three-axis coils 14-1 and 14-2 of the antenna 28 to receive the magnetic field in time series. That is, the controller and signal processor 22 functions as a control section that generates magnetic fields at different times from each of the plurality of magnetic field generators and causes the plurality of three-axis magnetic field detectors to detect the magnetic fields in time series. The magnetic field received by the three-axis coils 14-1 and 14-2 of the antenna 28 is calculated and processed at each time by the controller and signal processor 22 as a calculator, and a spatial position and/or a spatial direction of each of the one-axis coils 12 is determined. By quickly switching the one-axis coil 12 that transmits a magnetic field, the position and/or direction of each part of the insertion portion 38 of the endoscope 36 can be determined almost in real time, and the shape of the insertion portion 38 can be reproduced by joining the positions and/or directions.

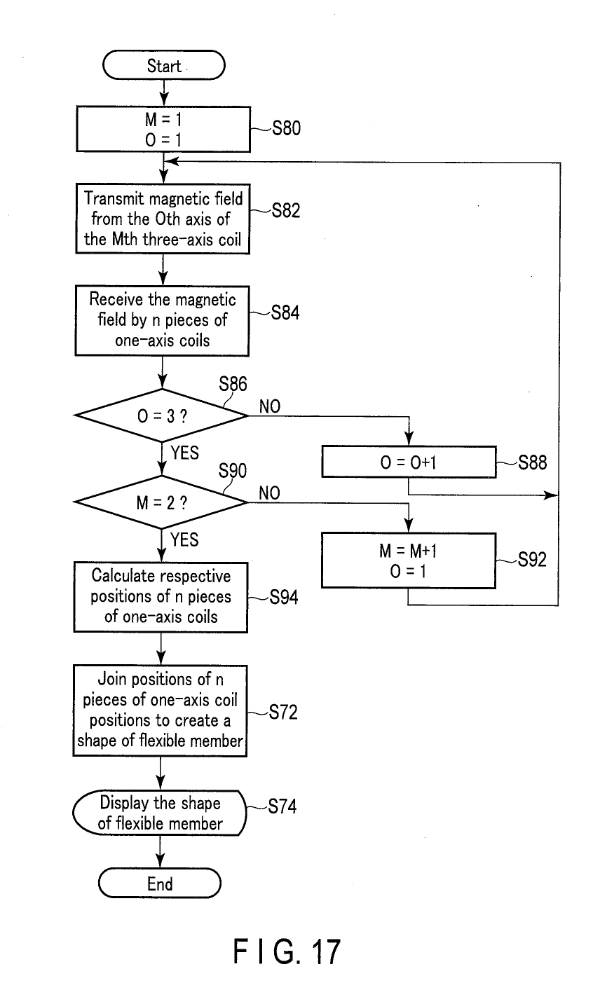

Also in this embodiment, the roles of the transmission side and the reception side can be exchanged. The operation of the magnetic field sensor system 10 in this case is as shown in the flowchart of FIG. 17.

That is, first, the controller and signal processor 22 initializes

a variable counter M and a variable counter O, which are constructed therein, respectively to 1 (step S80).

Thereafter, the controller and signal processor 22 generates a magnetic field from the Oth axis, i.e., a coil 26 which is the Oth one-axis magnetic field generation element of the Mth three-axis coil among the two three-axis coils 14-1 and 14-2 in the antenna 28, namely, causes the coil to transmit a magnetic field (step S82). The controller and signal processor 22 causes each of the n pieces of one-axis coils 12-1 to 12-n in the insertion section 38 of the endoscope 36 to detect a magnetic field, namely, courses them to receive a magnetic field, and then stores the respective detection results in an unillustrated internal memory (step S84). Then, the controller and signal processor 22 determines whether or not the value of the variable counter O is 3 (step S86). In this step, when it is determined that the value of the variable counter O has not yet reached 3, the controller and signal processor 22 increments the value of the variable counter O by 1 (step S88), and repeats the processing from step S82.

If it is determined in step S86 that the value of the variable counter O has reached 3, the controller and signal processor 22 determines whether or not the value of the variable counter M has reached 2 (step S90). In this step, if it is determined that the value of the variable counter M has not yet reached 2, the controller and signal processor 22 increments the value of the variable counter M by 1 and sets the value of the variable counter O to 1 (step S92), and then, repeats the processing from step S82.

In this way, the controller and signal processor 22 functions as a control section that causes each of a plurality of three-axis magnetic field generators to generate magnetic fields at different times and causes a plurality of magnetic field detectors to detect axial magnetic field components in time series.

If it is determined in step S90 that the value of the variable counter M has reached 2, the controller and signal processor 22 as a calculator calculates respective spatial positions and/or spatial directions of the n pieces of one-axis coils 12-1 to 12-n from the detection results of the magnetic fields stored in the internal memory (step S94). Thereafter, the controller and signal processor 22 joins the calculated spatial positions and/or spatial directions of the n pieces of one-axis coils 12-1 to 12-n to create flexible-member-shape information showing the shape of the insertion portion 38 (step S72). The flexible member shape information is displayed on a display device that displays an endoscopic image or another display device (step S74).

As described above, when a magnetic field is transmitted in time series from a specific axis of a specific three-axis coil, (one-axial components of) the magnetic fields at the specific times are acquired simultaneously by each of the n pieces of one-axis coils. Therefore, it is possible to obtain the position and/or the direction of a specific one-axis coil from the information on (the one-axial components of) the magnetic fields by each axis on the three-axis coil, the magnetic fields being acquired in time series by the specific one-axis coil.

In this case, by switching the transmission of a magnetic field as soon as possible, it is possible to regard the movement of the endoscope 36 during acquisition of all the signals as nearly 0, and to calculate the positions and/or directions with high accuracy. By joining the positions and/or directions, the shape of the endoscope 36 can be reproduced.

As described in the first embodiment, as a matter of course, the two-axis coils 34 may be used instead of the one-axis coils 12-1 to 12-n.

In the above description, the case is described where the antenna 28 incorporates two three-axis coils 14-1 and 14-2; however, as a matter of course, three or more three-axis coils may be incorporated into the antenna 28 as in the second embodiment.

In the actual use state of the endoscope 36, as shown in FIG. 18, a patient 44 having an intestinal tract, which is a tubular object to be inspected into which the insertion portion 38 is inserted, is placed on a bed 46, and an operator 48 such as a doctor will operate the endoscope 36. An operation section 40 of the endoscope 36 is connected to a main body 50 via a cable 42. At this time, the antenna incorporating the three-axis coils is disposed at a position that does not interfere with the operation of the operator 48 as much as possible, but since the detectable range of a magnetic field is limited, the antenna cannot be separated far from the bed 46.

At this time, if three three-axis coils are arranged on a plane as disclosed in the above-mentioned International Publication No. 94/04938, it is inevitable to use a plane antenna 52, causing restrictions on the surrounding operation of the operator 48.

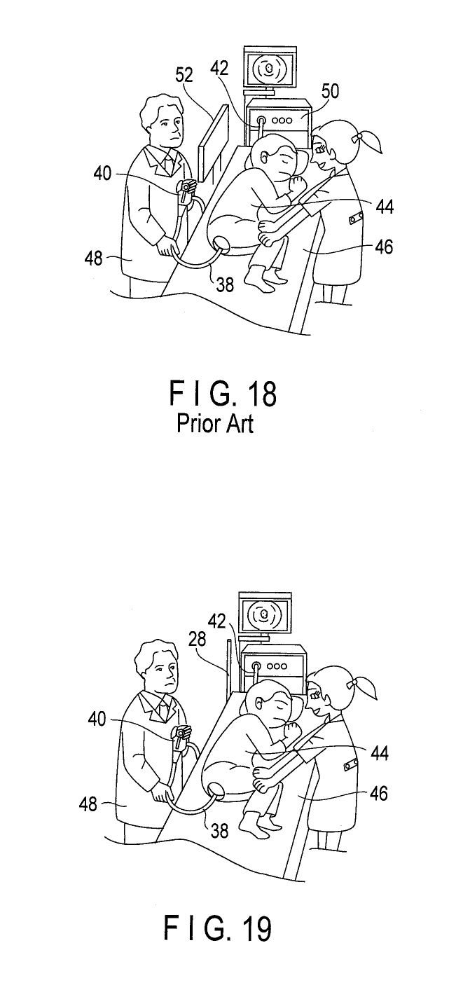

In contrast, as described in the first and second embodiments, the antenna 28, in which two, three or more three-axis coils are arranged on a substantially straight line, can be configured as a rod-shaped antenna. That is, the antenna 28 in which two, three or more three-axis coils are arranged in the substantially vertical direction, namely, in the gravity direction, can be configured. Therefore, the range, in which the operator 48 such as a doctor can move, can be expanded, and the restrictions on the behavior of the operator 48 can be minimized, even if the antenna 28 is placed perpendicularly to the plane of the bed 46 and close to the bed 46 as shown in FIG. 19. In addition, since this arrangement allows the antenna 28 to be separated from the bed 46, it is possible to minimize the influence of distortion of a magnetic field caused by metal (for example, due to a metal frame of the bed 46) when using an AC magnetic field.

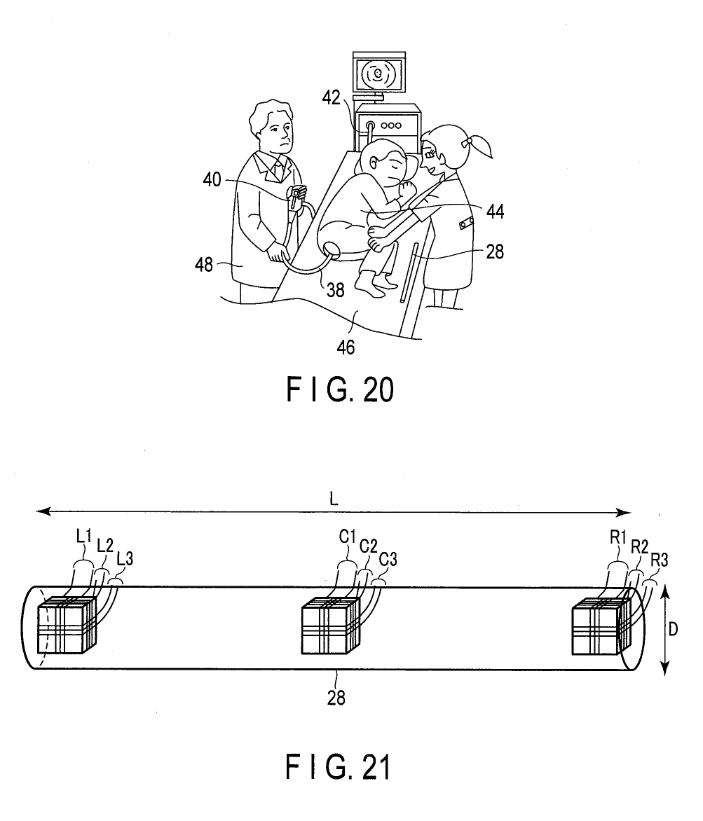

Furthermore, the antenna 28 in which two, three or more three-axis coils are arranged in a substantially horizontal direction, namely, in a direction perpendicular to the gravity direction, can be configured. The antenna 28 can be placed adjacent to the bed 46; for example, it could be placed on the plane of the bed 46 as shown in FIG. 20, minimizing the restrictions on the behavior of the operator 48 such as a doctor. This configuration is useful when the bed 46 is made of a nonmetal.

In the arrangement as shown in FIGS. 19 and 20, the bar-shaped antenna 28 using the three-axis coils in combination can be effectively used because it does not restrict the behavior of the operator 48 such as a doctor, if an L/D is 5 or more, as shown in FIG. 21. Here, “L” is a longest distance of a distribution of the three-axis coils in the straight-line axial direction arranged on a straight line, and “D” is a distance in the perpendicular direction thereto. In brief, “L” is a size in the longitudinal direction of the antenna 28, and “D” can be regarded as a width in a direction orthogonal to the longitudinal direction of the antenna 28, i.e., a diameter of the antenna 28, in this example. It is more effective to set the L/D to 10 or more.

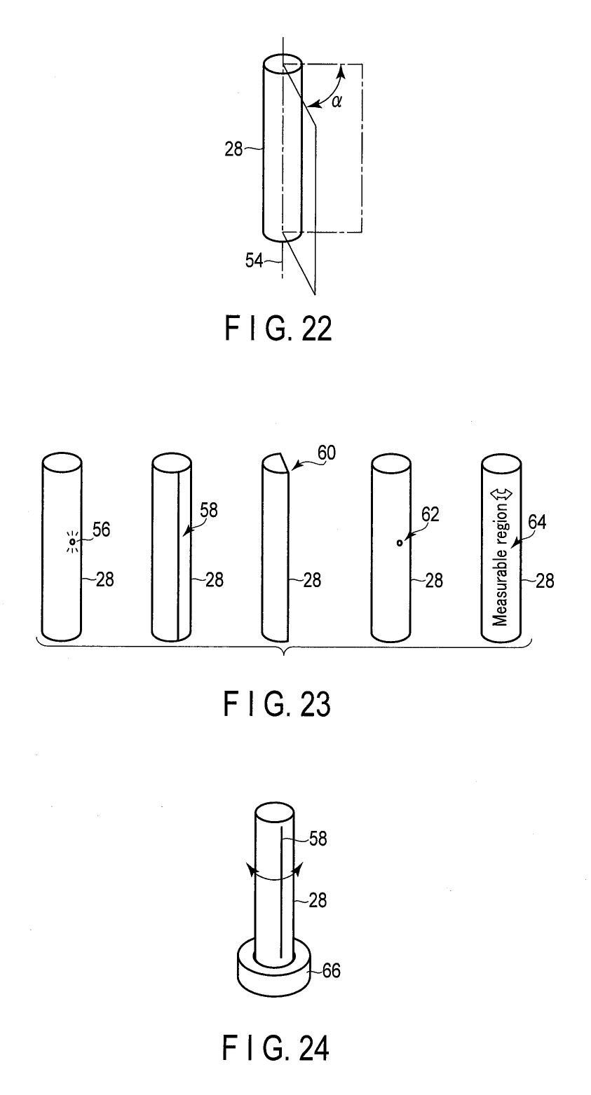

Incidentally, since the antenna 28 is in the shape of a rod, for example, a columnar shape, there is a degree of freedom in the way of installation in the circumferential directions thereof. When the Gauss-Newton method, etc., is used, for example, as a position detection algorithm, a position detectable region exists depending on how to take an initial value (vector). Generally, a position detection algorithm has a position detectable region. Therefore, as shown in FIG. 22, since the antenna 28 has a rod-shaped shape, the antenna 28 can be freely disposed with respect to the rotational direction using a straight line (line segment) 54, in which two, three or more three-axis coils are arranged, as a rotation axis. It is necessary to direct the direction of the antenna 28 including a specific position-detectable rotation angle α to a detection target portion of the insertion section 38.

Therefore, it is desirable to provide an identification section for identifying a position detectable region in the antenna 28 as shown in FIG. 23. For example, any identification section may be used, such as, a pilot lamp 56, a mark indicating a direction such as a line 58, a feature of a shape (formation of a convex shape or a corner portion 60, etc.), a pin 62, letters 64, etc., as long as the direction in which the position detectable region should be directed to a detection target portion can be discriminated by the identification section.

As described above, by identifying and displaying the center of a position detectable region or an angular direction corresponding to the position detectable region, it is possible to set the rod-shaped antenna 28 in an optimum direction with respect to the detection target portion.

Here, a holder 66 that rotatably holds the rod-shaped antenna 28 in a substantially vertical direction may be provided to hold the antenna 28 such that the position detectable region can be easily directed to a desired direction as shown in FIG. 24. With such a configuration, it is possible to make the antenna 28 rotatable in a rigid body (namely, with the same angle as a whole) about a straight line (line segment) 54 on which the three-axis coils are arranged, and to smoothly install the antenna 28 at an optimum position.

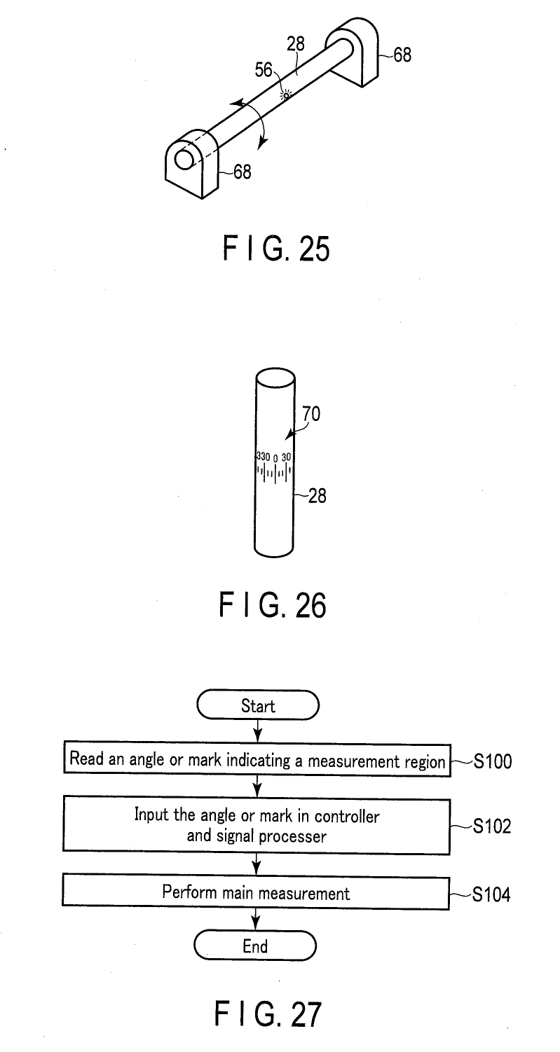

Alternatively, as shown in FIG. 25, the antenna 28 may be held by a holder 68 that holds the rod-shaped antenna 28 rotatably in a substantially horizontal direction.

As the identification section, it is also possible to provide the antenna 28 with a notation and a scale 70 indicating an angle as shown in FIG. 26, or to provide a unique mark for each angle. In this case, instead of directing the position-detectable region to a detection target portion by rotating the antenna 28, the position detection algorithm is changed without moving the antenna 28 itself so that the position can be detected.

That is, as shown in FIG. 27, the operator 48 reads the notation and scale 70 or a mark indicating the angle of the direction in which a detection target portion exists (step S100), and inputs the result in the controller and signal processor 22 by using an unillustrated input section (step S102). Thereafter, the main measurement as described in the first to third embodiments is performed (step S104). In this main measurement, for example, in the case of using the Gauss-Newton method, etc., the position and/or direction of the one-axis coil 12 can be calculated in a region where a detection target portion exists by optimizing the way of taking the initial value in the designated direction.

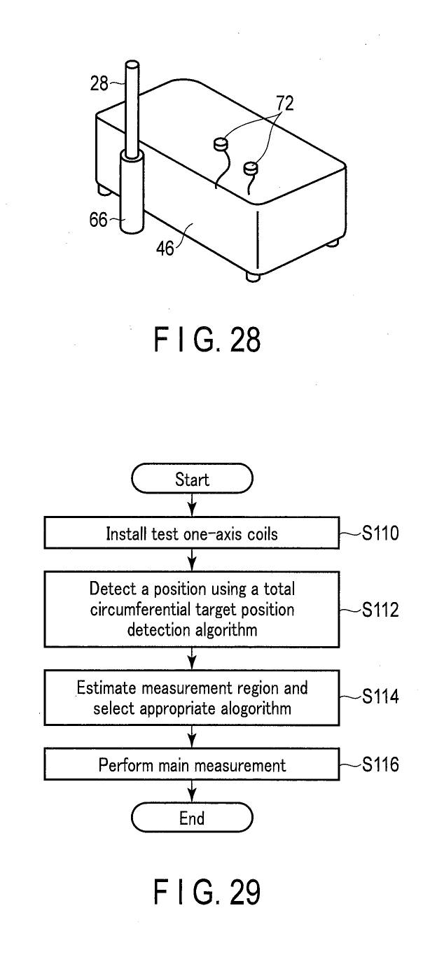

Furthermore, instead of causing the operator 48 to rotate the antenna 28 and input the notation and scale 70 or a mark indicating an angle, it is also possible to automate a magnetic field sensor system by using a test one-axis coil. In the automation, as shown in FIG. 28, before an actual measurement, one or a plurality of test one-axis coils 72 are installed in a measurement region where it is assumed that a detection target portion is placed. The controller and signal processor 22 performs position detection using a position detection algorithm targeting the entire area from a magnetic field signal therefrom (or a magnetic field signal thereto) and judges the measurement region from information on the detection result to thereby select a position detection algorithm suitable for the measurement region.

In this case, as shown in the flowchart of FIG. 29, the operator 48 installs one or a plurality of test one-axis coils 72 and inputs predetermined start instructions in the controller and signal processor 22 from an unillustrated input unit (Step S110). In response to this, the controller and signal processor 22 performs the position detection by a total circumferential target position detection algorithm (step S112). In this total circumferential target position detection algorithm, for example, in the Gauss-Newton method, a calculation is repeatedly performed to search for a minimum value of S for a plurality of initial values with respect to a measured signal and to output an estimate for the respective initial values. Thereafter the values of S are compared with values obtained at respective positions and in respective directions to determine a position, and a direction having a smallest S is determined as a final estimate among them.

Then, the controller and signal processor 22 estimates a measurement region according to the determined position and direction, and selects an appropriate position detection algorithm (step S114). Thereafter, the operator 48 removes the one or more test one-axis coils 72 and then performs a main measurement as described in the first to third embodiments (step S116).

As the position detection algorithm for all regions requires more calculation time as described above, it is not practical to do it every time. Therefore, before an actual measurement, a position detection algorithm is set corresponding to an appropriate measurement region by performing a measurement with the test one-axis coils 72, which are test one-axis magnetic field generators or detectors. With such an operation, it is possible to perform the actual measurement with a shorter calculation time. As a matter of course, the one-axis coil 12 used for the main measurement may be used as the test single-axis coil 72.

As described above, the magnetic field sensor system according to the third embodiment can be provided to a flexible device such as an endoscope.

That is, the flexible device is a flexible device including a magnetic field sensor system as described in the first or second embodiment and a flexible member such as the insertion portion 38 of the endoscope 36. The one-axis or two-axis magnetic field generators or the one-axis or two-axis magnetic field detectors are magnetic field generators. A plurality of the magnetic field generators are arranged in the flexible member. The plurality of three-axis magnetic field detectors or three-axis magnetic field generators are a plurality of three-axis magnetic field generators. The flexible device further includes a controller and signal processor 22 which is a controller configured to cause each of the plurality of magnetic field generators to generate a magnetic field therefrom at different times from each other and to cause the plurality of three-axis magnetic field detectors to detect a magnetic field in time series. The calculator is configured to calculate at least one of a position and a direction of each of the plurality of magnetic field generators, based on time-series detection results obtained by the plurality of three-axis magnetic field detectors.

Alternatively, the flexible device may be a flexible device including a magnetic field sensor system as described in the first or second embodiment and a flexible member such as the insertion portion 38 of the endoscope 36, wherein the one-axis or two-axis magnetic field generators or the one-axis or two-axis magnetic field detectors are one-axis or two-axis magnetic field detectors. A plurality of the magnetic field detectors are arranged in the flexible member. The plurality of three-axis magnetic field detectors or three-axis magnetic field generators are a plurality of three-axis magnetic field generators. The flexible device further includes a controller and signal processor 22 which is a controller configured to cause each of the plurality of three-axis magnetic field generators to generate a magnetic field therefrom at different times from each other and to cause the plurality of magnetic field detectors to detect an axial magnetic field component in time series. The calculator is configured to calculate at least one of a position and a direction of each of the plurality of magnetic field generators, based on a time-series detection results of axial direction magnetic field components obtained by the plurality of magnetic field detectors.

It should be noted that the plurality of three-axis magnetic field detectors or generators are arranged such that the centers of gravity of all the axes of magnetic field detection elements or magnetic field generation elements are respectively placed at a position, in a direction perpendicular to the longest distance, which is ⅕, more preferably 1/10 of the longest distance of a distribution of the magnetic field detection elements or magnetic field generation elements being arranged on a substantially straight line in a straight-line axis direction.

With such a configuration, it is effective in order not to restrict the behavior of the operator 48 such as a doctor.

In addition, the plurality of three-axis magnetic field detectors or generators can be arranged in a substantially gravity direction.

With such a configuration, even if the antenna 28 is placed adjacent to a bed 46, the range in which the operator 48 such as a doctor can move is expanded, and the restrictions on behavior of the operator 48 can be minimized. In the case where the plurality of three-axis magnetic field detectors or generators are arranged in the substantially gravity direction, they are arranged, for example, in such a manner that all three-axis magnetic field detectors or generators other than the uppermost three-axis magnetic field generator or detector among the plurality of three-axis magnetic field detectors or generators are located in a region within 20 degrees from the gravity direction with reference to the gravity of the uppermost three-axis magnetic field generator or detector.

Alternatively, the plurality of three-axis magnetic field detectors or generators may be arranged in a direction substantially perpendicular to the gravity direction.

With such a configuration, the antenna 28 can be placed adjacent to the bed 46, and the restrictions on behavior of the operator 48 such as a doctor can be minimized. In the case where the plurality of three-axis magnetic field detectors or generators are arranged in the direction substantially perpendicular to the gravity direction, they are arranged, for example, such that all three-axis magnetic field detectors or generators other than a three-axis magnetic field detector or generator located at the extreme end among the plurality of three-axis magnetic field detectors or generators are located in a region within 20 degrees from the direction perpendicular to the gravity direction with reference to the gravity of the three-axis magnetic field detector or generator located at the extreme end.

The calculator can calculate at least one of a spatial position and a spatial direction of the one-axis or two-axis magnetic field generator or the one-axis or two-axis magnetic field detector in a region including a specific rotation angle with respect to a straight line axis placed on the straight line. The plurality of magnetic field detectors or generators are housed in a common exterior, and the exterior may be provided with an identification section for identifying the specific rotation angle direction. Here, the identification section includes marks and shapes such as a pilot lamp 56, a mark indicating a direction such as a line 58, a feature of a shape (formation of a convex shape or a corner portion 60, etc.), a pin 62, letters 64, etc.

In this way, by displaying the center of a position-detectable region or an angular direction corresponding to the position-detectable region so that it can be identified, it is possible to set the rod-shaped antenna 28 in an optimum direction with respect to a detection target portion.

In this case, a holder 66 or 68 that rotatably holds the plurality of magnetic field detectors or generators and the exterior with respect to the straight line axis as a rotation axis may further be provided.

With this, it is possible to easily set the rod-shaped antenna 28 in an optimum direction with respect to a detection target portion.

Furthermore, the calculator can calculate a spatial position of the one-axis or two-axis magnetic field generator or detector for a region including the total angle of rotation with respect to a straight line axis placed on the straight line. The calculator may be configured such that if the position is calculated for the region including the total angle of rotation, the subsequent position calculation may be performed for a region including a specific rotation angle including the position.

By adopting such a configuration, it is possible to detect the position by changing the position detection algorithm without moving the antenna 28 itself, by not directing a position detectable region to a detection target portion with rotating the antenna 28.

### Modification

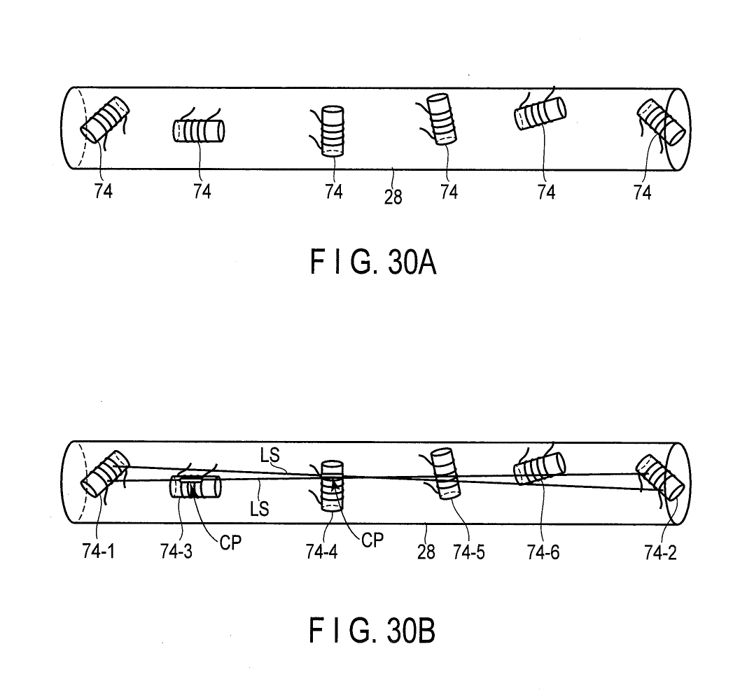

The first to third embodiments described above can be realized, even when a plurality of one-axis coils 74 as one-axis magnetic field detectors are arranged on a substantially straight line in the exterior of the antenna 28 as shown in FIG. 30A. That is, according to this configuration, for example, since the magnetic field generator to be disposed inside the insertion portion 38 of the endoscope 36 requires only the one-axis coil 12, it is possible to downsize and reduce the diameter. Furthermore, since the magnetic field detectors outside the endoscope 36 are also arranged in a one-dimensional rod shape, they do not become an impediment to the operator 48.

From the position calculation method described above, the number of the one-axis coils 74, which are one-axis magnetic field detectors, needs to be six or more. If the direction vectors of the six or more one-axis coils 74 are set to include three directional vectors that are linearly independent from each other, it is possible to further improve the accuracy of the position calculation.

It is even better if the one-axis coils 74, which are a plurality of one-axis magnetic field detectors, are arranged as shown in FIG. 30B. That is, a first one-axis coil 74-1 and a second one-axial coil 74-2 can be selected, and there exists certain line segments LS for each of the one-axis coils connecting the magnetic field detection/generation region of the first one-axis coil 74-1 and the magnetic field detection/generation region of the second one-axis coil 74-2. The other one-axis coils 74-3 to 74-6 are arranged in such a manner that their magnetic field detection/generation regions intersect with this line segments LS, namely, so as to have intersection portions CP.

With this configuration, the antenna 28 has a shape that reduces interference with the operator 48 even further.

The transmission side and the detection side described above may be reversed is the same as the embodiments of the present application.

Furthermore, the fact that the two-axis coil 34 may be used in place of the one-axis coil 12 is also the same as the embodiments of the present application.

Additional advantages and modifications will readily occur to those skilled in the art. Therefore, the invention in its broader aspects is not limited to the specific details, and representative devices shown and described herein.

Accordingly, various modifications may be made without departing from the spirit or scope of the general inventive concept as defined by the appended claims and their equivalents.

What is claimed is:

1. A magnetic field sensor system comprising:
one of a one-axis magnetic field generator and a two-axis magnetic field generator that generates a magnetic field;
three or more three-axis magnetic field detectors that detect a magnetic field for each axis and are arranged on a substantially straight line; and
a calculator that calculates, from a detection result of the magnetic field, at least one of a spatial position and a spatial direction of the one of the one-axis magnetic field generator and the two-axis magnetic field generator,
wherein the calculator is configured to
select two or more three-axis magnetic field detectors, which generate no symmetry of magnetic fields, from among the three or more three-axis magnetic field detectors, based on a preset judgment standard of symmetry of magnetic fields, and
calculate the at least one of the spatial position and the spatial direction of the one of the one-axis magnetic field generator and the two-axis magnetic field generator, based on detection results of the magnetic field between the selected two or more three-axis magnetic field detectors and the one of the one-axis magnetic field generator and the two-axis magnetic generator.

2. The magnetic field sensor system according to claim 1, wherein
the three or more three-axis magnetic field detectors respectively include magnetic field detection elements for each axis,
the magnetic field elements respectively have a center of gravity,
the respective magnetic field detection elements of the three or more three-axis magnetic field detectors are arranged on a substantially straight line, and
the three or more three-axis magnetic field detectors are arranged in such a manner that a longest distance of a distribution of centers of gravity of the magnetic field detection elements in a perpendicular direction to a straight-line axis direction of the substantially straight line becomes ⅕ of a longest distance of a distribution of the centers of gravity of the magnetic field detection elements in the straight-line axis direction.

3. The magnetic field sensor system according to claim 1, wherein
the three or more three-axis magnetic field detectors respectively include magnetic field detection elements for each axis,
the magnetic field elements respectively have a center of gravity,
the respective magnetic field detection elements of the three or more three-axis magnetic field detectors are arranged on a substantially straight line, and
the three or more three-axis magnetic field detectors are arranged in such a manner that a longest distance of a distribution of centers of gravity of the magnetic field detection elements in a perpendicular direction to a straight-line axis direction of the substantially straight line becomes 1/10 of a longest distance of a distribution of the centers of gravity of the magnetic field detection elements in the straight-line axis direction.

4. The magnetic field sensor system according to claim 1, wherein the three or more three-axis magnetic field detectors are arranged in a substantially gravity direction.

5. The magnetic field sensor system according to claim 1, wherein the three or more three-axis magnetic field detectors are arranged in a substantially perpendicular direction to a gravity direction.

6. The magnetic sensor system according to claim 1, wherein
the calculator is configured to
select the two or more three-axis magnetic field detectors which generate no symmetry of magnetic fields from among the three or more three-axis magnetic field detectors, based on the preset judgment standard of symmetry of magnetic fields regarding a value of the magnetic field measured by the two or more three-axis magnetic field detectors, and
calculate the at least one of the spatial position and the spatial direction of the one-axis magnetic field generator, based on detection results of the selected two or more three-axis magnetic field detectors.

7. The magnetic sensor system according to claim 1, wherein
the three or more three-axis magnetic field detectors respectively have a magnetic field detection region,
the three or more three-axis magnetic field detectors include a first three-axis magnetic field detector and a second three-axis magnetic field detector, and
the three-axis magnetic field detectors other than the first and second three-axis magnetic field detectors are arranged in such a manner that respective magnetic field detection regions thereof intersect with a line segment connecting the magnetic field detection region of the first three-axis magnetic field detector and the magnetic field detection region of the second three-axis magnetic field detector.

8. The magnetic sensor system according to claim 1, wherein
the three or more three-axis magnetic field detectors respectively include magnetic field detection elements for each axis,
the magnetic field detection elements respectively have a magnetic field detection region,
the three or more three-axis magnetic field detectors include a first three-axis magnetic field detector and a second three-axis magnetic field detector, and
of the three or more three-axis magnetic field detectors, magnetic field detection elements of each axis other than a one-axis magnetic field detection element in the first three-axis magnetic field detector and a one-axis magnetic field detection element in the second three-axis magnetic field detector are arranged in such a manner that respective magnetic field detection regions thereof intersect with a line segment connecting the magnetic field detection region of the one-axis magnetic field detection element of the first three-axis magnetic field detector and the magnetic field detection region of the one-axis magnetic field detection element of the second three-axis magnetic field detector.

9. The magnetic field sensor system according to claim 1, wherein
the calculator is capable of calculating the at least one of the spatial position and the spatial direction of the one of the one-axis magnetic field generator and the two-axis magnetic field generator in a region including a specific rotation angle about a straight line axis placed on the substantially straight line,
the three or more three-axis magnetic field detectors are housed in a common exterior, and
the exterior comprises an identification section for identifying the specific rotation angle.

10. The magnetic field sensor system according to claim 9, further comprising:
a holder that rotatably holds the exterior and the three or more three-axis magnetic field detectors, using the straight line axis as a rotation axis.

11. The magnetic field sensor system according to claim 1, wherein
the calculator is capable of calculating the spatial position of the one of the one-axis magnetic field generator and the two-axis magnetic field generator in a region including total angle of rotation about a straight line axis placed on the substantially straight line, and
if the position is calculated in the region including the total angle of rotation, the calculator is configured to perform subsequent position calculations for a region including a specific rotation angle including the position.

12. A flexible device comprising:
the magnetic field sensor system according to claim 1, and
a flexible member, wherein
the flexible member includes one of a plurality of one-axis magnetic field generators and a plurality of two-axis magnetic field generators,
the flexible device further comprises a controller configured to generate a magnetic field from each of the plurality of the magnetic field generators at different times from each other and to cause the three or more three-axis magnetic field detectors to detect a magnetic field in time series, and
the calculator is configured to calculate at least one of a spatial position and a spatial direction of each of the plurality of magnetic field generators, based on time-series detection results obtained by the three or more three-axis magnetic field detectors.

13. A magnetic field sensor system comprising:
one of a one-axis magnetic field detector and a two-axis magnetic field detector that detects a magnetic field;
three or more three-axis magnetic field generators that generate a magnetic field for each axis and are arranged on a substantially straight line; and
a calculator that calculates, from a detection result of the magnetic field, at least one of a spatial position and a spatial direction of the one of the one-axis magnetic field detector and the two-axis magnetic field detector,
wherein the calculator is configured to
select two or more three-axis magnetic field generators, which generate no symmetry of magnetic fields, from among the three or more three-axis magnetic field generators, based on a preset judgment standard of symmetry of magnetic fields, and
calculate the at least one of the spatial position and the spatial direction of the one of the one-axis magnetic field detector and the two-axis magnetic field detector, based on detection results of the magnetic field between the selected two or more three-axis magnetic field generators and the one of the one-axis magnetic field detector and the two-axis magnetic detector.

14. The magnetic field sensor system according to claim 13, wherein
the three or more three-axis magnetic field generators respectively include magnetic field generation elements for each axis,
the magnetic field generation elements respectively have a center of gravity,
the respective magnetic field generation elements of the three or more three-axis magnetic field generators are arranged on a substantially straight line, and
the three or more three-axis magnetic field generators are arranged in such a manner that a longest distance of a distribution of centers of gravity of the magnetic generation elements in a perpendicular direction to a straight-line axis direction of the substantially straight line becomes ⅕ of a longest distance of a distribution of the centers of gravity of the magnetic generation elements in the straight-line axis direction.

15. The magnetic field sensor system according to claim 13, wherein
the three or more three-axis magnetic field generators respectively include magnetic field generation elements for each axis,
the magnetic field generation elements respectively have a center of gravity,
the respective magnetic field generation elements of the three or more three-axis magnetic field generators are arranged on a substantially straight line, and
the three or more three-axis magnetic field generators are arranged in such a manner that a longest distance of a distribution of centers of gravity of the magnetic generation elements in a perpendicular direction to a straight-line axis direction of the substantially straight line becomes 1/10 of a longest distance of a distribution of the centers of gravity of the magnetic generation elements in the straight-line axis direction.

16. The magnetic field sensor system according to claim 13, wherein the three or more three-axis magnetic field generators are arranged in a substantially gravity direction.

17. The magnetic field sensor system according to claim 13, wherein the three or more three-axis magnetic field generators are arranged in a substantially perpendicular direction to a gravity direction.

18. The magnetic sensor system according to claim 13, wherein
wherein the calculator is configured to
individually generate magnetic fields for each axis by the three or more three-axis magnetic field generators,
measure magnetic fields by the one of the one-axis magnetic field detector and the two-axis magnetic field detector;
select the two or more three-axis magnetic field generators which generate no symmetry of magnetic fields from among the three or more three-axis magnetic field generators, based on the magnetic fields measured by the one of the one-axis magnetic field detector and the two-axis magnetic field detector and the preset judgment standard of symmetry of magnetic fields; and
calculate the at least one of the spatial position and the spatial direction of the one of the one-axis magnetic field detector and the two-axis magnetic field detector, based on detection results of the one of the one-axis magnetic field detector and the two-axis magnetic field detector for the magnetic field individually generated for each axis by the selected two or more three-axis magnetic field generators.

19. The magnetic sensor system according to claim 13, wherein
the three or more three-axis magnetic field generators respectively have a magnetic field generation region,
the three or more three-axis magnetic field generators include a first three-axis magnetic field generator and a second three-axis magnetic field generator, and
the three-axis magnetic field generators other than the first and second three-axis magnetic field generators are arranged in such a manner that respective magnetic field generation regions thereof intersect with a line segment connecting the magnetic field generation region of the first three-axis magnetic field generator and the magnetic field generation region of the second three-axis magnetic field generator.

20. The magnetic sensor system according to claim 13, wherein
the three or more three-axis magnetic field generators respectively include magnetic field generation elements for each axis,
the magnetic field generation elements respectively have a magnetic field generation region,
the three or more three-axis magnetic field generators include a first three-axis magnetic field generator and a second three-axis magnetic field generator, and
of the three or more magnetic field generators, magnetic field generation elements of each axis other than a one-axis magnetic field generation element in the first three-axis magnetic field generator and a one-axis magnetic field generation element in the second three-axis magnetic field generator are arranged in such a manner that respective magnetic field generation regions thereof intersect with a line segment connecting the magnetic field generation region of the one-axis magnetic generation element of the first three-axis magnetic field generator and the magnetic field generation region of the one-axis magnetic field generation element of the second three-axis magnetic field generator.

21. The magnetic field sensor system according to claim 13, wherein
the calculator is capable of calculating the at least one of the spatial position and the spatial direction of the one of the one-axis magnetic field detector and the two-axis magnetic field detector in a region including a specific rotation angle about a straight line axis placed on the substantially straight line,
the three or more three-axis magnetic field generators are housed in a common exterior, and
the exterior comprises an identification section for identifying the specific rotation angle.

22. The magnetic field sensor system according to claim 21, further comprising:
a holder that rotatably holds the exterior and the three or more three-axis magnetic field generators, using the straight line axis as a rotation axis.

23. The magnetic field sensor system according to claim 13, wherein
the calculator is capable of calculating the spatial position of the one of the one-axis magnetic field generator and the two-axis magnetic field generator in a region including total angle of rotation about a straight line axis placed on the substantially straight line, and
if the position is calculated in the region including the total angle of rotation, the calculator is configured to perform subsequent position calculations for a region including a specific rotation angle including the position.

24. A flexible device comprising:
the magnetic field sensor system according to claim 13, and
a flexible member, wherein
the flexible member includes one of a plurality of one-axis magnetic field detectors and a plurality of two-axis magnetic field detectors,
the flexible device further comprises a controller configured to generate a magnetic field from each of the three or more tree-axis magnetic field generators at different times from each other and to cause the plurality of magnetic field detectors to detect an axial magnetic field component in time series, and
the calculator is configured to calculate at least one of a spatial position and a spatial direction of each of the plurality of magnetic field detectors, based on a detection result of time-series axial-direction magnetic field components obtained by the plurality of magnetic field detectors.
